(19) United States
(12) Patent Application Publication
(10) Pub. No.: US 2023/0380438 A1
(43) Pub. Date: Nov. 30, 2023

(54) Native whey protein composition for improving gastro-intestinal tolerance

(71) Applicant: N.V. Nutricia, Zoetermeer (NL)

(72) Inventors: Ingrid Brunhilde Renes, Utrecht (NL); Evan Abrahamse, Utrecht (NL); Gerrit Hols, Utrecht (NL)

(73) Assignee: N.V. Nutricia, Zoetermeer (NL)

(21) Appl. No.: 18/250,228

(22) PCT Filed: Oct. 27, 2021
(86) PCT No.: PCT/EP2021/079738

§ 371 Date: Apr. 24, 2023

(30) Foreign Application Priority Data
Oct. 27, 2020 (WO) PCT/EP2020/080198

(51) Int. Cl. A23C 21/06; A23C 9/142; A23L 33/19; A23L 33/00

(52) U.S. Cl. CPC A23C 21/06; A23C 9/1422; A23L 33/19; A23L 33/40

(57) ABSTRACT

The invention concerns a native whey protein composition with profound amounts of native whey protein and with beta-casein for use in the treatment and/or prevention of gastrointestinal intolerance. The inventors found that the native whey protein composition relatively high in beta-casein and low in kappa- and alpha-casein provides a beneficial effect on gastrointestinal intolerance.

### FIELD OF THE INVENTION

The present invention relates to the field of compositions for use in the treatment and/or prevention of impaired gastro-intestinal tolerance, particularly in infants. Further disclosed are methods and uses for improved intestinal transit improved gastro-intestinal tolerance and/or digestive coagulation in the upper gastrointestinal tract in a subject.

### BACKGROUND

Human milk is the uncontested gold standard concerning infant nutrition. However, in some cases breastfeeding is inadequate or unsuccessful for medical reasons or because of a choice not to breastfeed. For such situations infant or follow on formulas have been developed. Commercial infant formulas are commonly used today to provide supplemental or sole source of nutrition early in life. Commercial infant formulas are designed to mimic, as closely as possible, the composition and function of human milk. To obtain infant formulas, processing of bovine milk is required to ensure microbial safety and to more accurately mimic the composition of human milk, in particular the ratio of whey:casein proteins as found in animal milk is adapted since the whey:casein ratio in human milk is different from bovine milk with the whey to casein ratio in human milk being about 60:40 compared to 20:80 in bovine milk [de Wit, J. dairy science, 1998, 81, 597-608]. Human milk also comprises less minerals, mainly beta-casein, less κ-casein and low amounts of α-casein as compared with bovine milk [Lonnerdal, 2003, 77, 1537s-1543s].

Feeding intolerance or gastrointestinal intolerance is frequently observed in very young infants, premature infants and infants that have a low birth weight or are small for gestational age due to immaturity of the gastrointestinal (GI) tract, and can result in secondary problems like malabsorption, impaired growth. Such gastrointestinal intolerance is more frequently observed in premature infants fed classical infant formulae than breast milk [Alarcon, Nutrition, 2002, 18, 484-489]. Milk processing can influence protein quality, digestibility, gastric emptying and GI transit time and thereby cause GI tolerance. A mild milk processing method as described in WO2019/160402A1 and WO2018/028764A1 provides for a native whey protein composition comprising native whey protein and beta-casein.

The present invention provides in the need in the art for a protein composition to be used to treat and/or prevent gastrointestinal intolerance and improve gastrointestinal tolerance, which is compatible with the regular enteral feeding regime and suitable for inclusion in infant formulae.

### SUMMARY OF THE INVENTION

While whey protein is commonly perceived being a ‘fast protein’, casein is a ‘slow protein’ that coagulates under stomach conditions. On the other hand, at least for humans, casein is the highest standard in terms of nutritional value. On many occasions, such as is the case with infant and follow-on formula, it is not a viable option to improve gastric emptying and reducing stomach coagulation by leaving out caseins. The inventors found a way to reduce coagulation behavior for a whey protein composition comprising whey protein but also significant amounts of (beta-)casein.

The inventors compared gastrointestinal tract tolerance upon feeding the native whey protein composition comprising beta-casein and native whey protein which were subjected to mild heat treatment (hereafter: ‘the native whey protein composition according to the invention’), and an extensively heat treated whey protein composition comprising the same amount of beta-casein and whey protein, in a preclinical piglet model. The inventors surprisingly found that the native whey protein composition which was subjected to mild heat treatment according to the invention significantly improved gastrointestinal tolerance in the preclinical piglet. The native whey protein composition according to the invention improved gastric emptying, increased the rate of gastric emptying, and reduced the rate and extent of gastric coagulation. The native whey protein composition according to the invention also improved intestinal transit.

Piglets are widely recognized in the field being a suitable non-human model system for studying the gastro-intestinal tract and drawing conclusions about the GI tract in human infants. Maturation of the intestines in piglets closely resembles that of human neonates and infants, and, in contrast to rodents, the postnatal gut development and nutritional requirements of piglets more closely resemble the human infant in many ways. Hence, based on the above findings, there is an imminent use of the mildly heat treated native whey protein composition for treating or preventing gastrointestinal issues in infants, particularly in preterms. The heat treated native whey protein composition is typically comprised in a nutritional composition, preferably an infant formula such as a preterm formula.

The present invention thus relates to a native whey protein composition comprising beta-casein and native whey protein, for use in the treatment and/or prevention of gastrointestinal intolerance, wherein the native whey protein composition has been pasteurized. The invention also relates to the use of a native whey protein composition comprising beta-casein and native whey protein, in the manufacture of a product for the treatment and/or prevention of gastrointestinal intolerance, wherein the native whey protein composition has been pasteurized (prior to inclusion in the product). Worded differently, the invention relates to a (non-therapeutic) method for treating and/or preventing gastrointestinal intolerance, wherein the method comprises administering to a subject in need thereof a native whey protein composition comprising beta-casein and native whey protein, and wherein the native whey protein composition has been pasteurized. Herein, the term ‘prevention’ also implies reducing the risk of occurrence, particularly in a subject such as an infant or a preterm that is at imminent or increased risk of occurrence of gastrointestinal intolerance.

The invention also concerns a (non-therapeutic) method for administering a native whey protein composition for reducing the rate and/or extent of coagulation in the stomach; and/or increasing the rate of gastric emptying; and/or improving intestinal transit in a subject, preferably a subject that is not at imminent or at increased risk of gastrointestinal intolerance.

In the context of the invention, ‘coagulation’ means digestive coagulation or coagulation under human stomach conditions (i.e. stomach coagulation). Coagulation in the context of the invention does not refer to any shelf or storage stability issues which may be induced or associated with protein stability.

The treatment and/or prevention of gastrointestinal intolerance preferably means treating and/or preventing delayed or slow gastric emptying, preventing and/or reducing the rate and extent of coagulation in the stomach, increasing the rate of gastric emptying and/or improving intestinal transit. In one aspect, the use or method can be advantageously used to prevent or treat (reduce) reflux or regurgitation which is a common issue to particularly infants.

In a preferred embodiment, the whey proteins have a nativity of more than 80%, preferably more than 90%, more preferably more than 92%, more preferably more than 94%, more preferably more than 95%, and even more preferably more than 98% of all (non-hydrolyzed) whey protein. Preferably, substantially no non-native whey protein is present in the whey protein composition. In case the whey protein composition is comprised in a nutritional composition, this applies to all whey protein in the nutritional composition.

The native whey proteins are intact whey proteins. Intact means that the whey proteins have not been subjected to a hydrolysis step, i.e. are not hydrolyzed. Thus, substantially no non-intact whey protein is present as whey protein. In case the native whey protein composition is comprised in a nutritional composition, this applies to all whey protein in the nutritional composition. In this context, it is preferred that less than 5 wt %, more preferably less than 1 wt % of all whey protein encompassed in the nutritional composition is in a hydrolyzed form.

In one embodiment, the whey protein composition is pasteurized and comprised in an infant formula and the infant formula. The inventors of the present invention have surprisingly shown that an infant formula with native whey protein, obtained by or obtainable by the method present invention that includes a mild pasteurization step, can be used for the treatment and/or prevention of gastrointestinal intolerance.

The preferred target subject is a human infant.

### List of Preferred Clauses

1. A native whey protein composition comprising beta-casein and native whey protein, for use in the treatment and/or prevention of gastrointestinal intolerance, wherein the native whey protein composition has been pasteurized.

2. The native whey protein composition for use according to clause 1, comprising less than 6 wt %, preferably less than 4 wt %, more preferably less than 2 wt % of the sum of alpha-casein and kappa-casein, based on the total protein weight.

3. The native whey protein composition for use according to clauses 1 or 2, wherein the treatment and/or prevention of gastrointestinal intolerance comprises at least one of
(a) treatment and/or prevention of delayed or slow gastric emptying; and/or
(b) reduction of the rate and/or extent of coagulation in the stomach; and/or
(c) increase the rate of gastric emptying; and/or
(d) improvement of tolerance in the upper gastrointestinal tract; and/or
(e) improvement of intestinal transit.

4. The native whey protein composition for use according to any one of the preceding clauses, wherein regurgitation and/or reflux are prevented and/or treated.

5. The native whey protein composition for use according to any one of the preceding clauses, wherein the whey protein has a nativity of more than 80%, preferably more than 90%, more preferably more than 92%, preferably more than 94%, preferably more than 95%, even more than 98%.

6. The native whey protein composition for use according to any one of the preceding clauses, wherein the native whey protein composition has a protein solubility of more than 55%, preferably more than 60%, more preferably more than 65%, more than 68%, more than 70% or even more than 71% based on the total amount of protein in the native whey protein composition at acidic pH conditions, preferably at a pH of 4.5-4.7.

7. The native whey protein composition for use according to any of the preceding clauses, wherein the upper gastrointestinal tolerance is improved in a vulnerable subject, preferably an infant, more preferably an infant selected from preterm infants, infants that are small for gestational age, infants with a low birth weight, very young infants and/or infants suffering from reflux and/or mild regurgitation.

8. The native whey protein composition for use according to any one of the preceding clauses, wherein the native whey protein composition is obtainable by cold membrane-filtration based technology.

9. The native whey protein composition for use according to any one of the preceding clauses, wherein the native whey protein composition has been pasteurized at 72-74° C. for 15 to 30 seconds.

10. The native whey protein composition for use according to any one of the preceding clauses, wherein the native whey does not originate from acid whey or from sweet whey.

11. The native whey protein composition for use according to any one of the preceding clauses, wherein the native whey protein composition comprises native whey protein and beta-casein in the range of 85:15 and 55:45, preferably between 80:20 and 55:45, more preferably between and 60:40, more preferably between 80:20 and 70:30.

12. The native whey protein composition for use according to any one of the preceding clauses, wherein the native whey protein composition is obtainable by a process comprising:
(a) processing defatted milk into a casein stream, a whey protein stream and a lactose stream, by:
   (i) subjecting the defatted milk to a debacterialization treatment, to provide a debacterialized milk;
   (ii) subjecting the debacterialized milk originating from step (i) to cold microfiltration over a membrane capable of retaining casein and permeating whey proteins, to provide a casein stream as retentate and a permeate comprising whey protein and β-casein;
   (iii) fractionating the permeate originating from step (ii) into a whey protein stream comprising 1) whey protein and β-casein and 2) a lactose stream;
(b″) optionally spray-drying the whey protein stream originating from (a)-(iii) followed by dissolving;
wherein at least one of the debacterialization treatment of step (a)-(i) or the stream originating from step (a)-(iii) after optional step (b″) is subjected to pasteurization; and;
(c″) optionally freeze-drying the stream,
and wherein the debacterialization treatment (i) if not pasteurization, involves subjecting the defatted milk to microfiltration over a membrane capable of retaining bacteria, wherein the debacterialized milk is in the permeate.

13. The native whey protein composition for use according to clause 12, wherein step (iii) is performed by ultrafiltration over a membrane capable of retaining whey proteins and permeating lactose, to provide a whey protein stream as retentate and a permeate comprising lactose, preferably wherein ultrafiltration step (iii) operates with a volume concentration factor in the range of 20-200.

14. The native whey protein composition for use according to any one of the preceding clauses, wherein the native whey protein composition is part of a nutritional composition, preferably an infant formula or follow-on formula, more preferably a preterm formula and/or a formula for infants having a low birth weight.

15. The native whey protein composition for use according to clause 14, wherein the native whey protein composition and optionally at most 2 wt % of added free amino acids based on the total weight of protein in the nutritional composition are the sole protein sources for the nutritional composition.

16. The native whey protein composition for use according to any one of clauses 12-15, wherein the nutritional composition exhibits an alkaline phosphatase activity of at most 350 mU/L.

17. A nutritional composition comprising 3 to 7 g lipid/100 kcal, 1.25 to 5 g protein/100 kcal and 6 to 18 g digestible carbohydrate/100 kcal,
wherein the protein fraction consists of whey protein and beta-casein, and is substantially devoid of alpha-casein and kappa-casein, preferably comprising less than 6 wt %, preferably less than 4 wt %, more preferably less than 2 wt % of the sum of alpha-casein and kappa-casein, based on the total protein weight of the protein fraction, and wherein the formula optionally comprises added free amino acids,
wherein the ratio of whey protein to beta-casein is in the range of between 85:15 and 55:45, preferably between 80:20 and 55:45, more preferably between 80:20 and 60:40, more preferably between 80:20 and 70:30, wherein the protein fraction has been pasteurized, and wherein the whey protein has a nativity of more than 80%, preferably more than 90%, more preferably more than 92%, preferably more than 94%, preferably more than 95%, even more than 98% of the whey protein.

18. The nutritional composition according to clause 17 wherein the ratio beta-lactoglobulin to alpha-lactalbumin is below 7:3.

19. The nutritional composition according to any one of clauses 17 and 18 wherein the nutritional composition is selected from a preterm formula, an infant formula and a follow on formula.

20. A method for administering a native whey protein composition comprising beta-casein and whey protein for reducing the rate and/or extent of coagulation in the stomach; and/or
increasing the rate of gastric emptying; and/or improving intestinal transit in a subject, preferably a subject that is not at imminent or at increased risk of gastrointestinal intolerance, wherein the whey protein composition has been pasteurized, and wherein the whey protein has a nativity of more than 80%, preferably more than 90%, more preferably more than 92%, preferably more than 94%, preferably more than 95%, even more than 98%.

21. Use of a native whey protein composition comprising beta-casein and whey protein in the manufacture of a product for the treatment and/or prevention of gastrointestinal intolerance, wherein the native whey protein composition has been pasteurized, and wherein the whey protein has a nativity of more than 80%, preferably more than 90%, more preferably more than 92%, preferably more than 94%, preferably more than 95%, even more than 98%.

22. The native whey protein composition for use according to any one of clauses 1-11, wherein the native whey protein composition is obtainable by a process comprising either:
(a) processing defatted milk into a casein stream, a whey protein stream and a lactose stream, by:
   (i) subjecting the defatted milk to a debacterialization treatment, to provide a debacterialized milk;
   (ii) subjecting the debacterialized milk originating from step (i) to cold microfiltration over a membrane capable of retaining casein and permeating whey proteins, to provide a casein stream as retentate and a permeate comprising whey protein and β-casein;
   (iii) fractionating the permeate originating from step (ii) into a whey protein stream comprising 1) whey protein and β-casein and 2) a lactose stream;
(b″) optionally spray-drying the whey protein stream originating from (a)-(iii) followed by dissolving;
(b2) pasteurizing the whey protein stream originating from step (iii) or (b″);
(c″) optionally freeze-drying the whey protein stream originating from step (b2);
wherein step (a)(i) involves subjecting the defatted milk to microfiltration over a membrane capable of retaining bacteria and permeating milk proteins;
or,
(a) processing defatted milk into a casein stream, a whey protein stream and a lactose stream, by:
   (i) subjecting the defatted milk to a debacterialization treatment, to provide a debacterialized milk;
   (ii) subjecting the debacterialized milk originating from step (i) to cold microfiltration over a membrane capable of retaining casein and permeating whey proteins, to provide a casein stream as retentate and a permeate comprising whey protein and β-casein;
   (iii) fractionating the permeate originating from step (ii) into a whey protein stream comprising 1) whey protein and β-casein and 2) a lactose stream;
(b″) optionally spray-drying the whey protein stream originating from (a)-(iii) followed by dissolving;
(c″) optionally freeze-drying the whey protein stream originating from step (a) (iii) or (b″); wherein step (a) (i) involves pasteurizing the defatted milk.

23. Nutritional composition according to any one of clauses 17-19, wherein the nutritional composition is obtainable by a process comprising either:
(a) processing defatted milk into a casein stream, a whey protein stream and a lactose stream, by:
   (i) subjecting the defatted milk to a debacterialization treatment, to provide a debacterialized milk;
   (ii) subjecting the debacterialized milk originating from step (i) to cold microfiltration over a membrane capable of retaining casein and permeating whey proteins, to provide a casein stream as retentate and a permeate comprising whey protein and β-casein;
   (iii) fractionating the permeate originating from step (ii) into a whey protein stream comprising 1) whey protein and β-casein and 2) a lactose stream;
(b) combining at least part of the whey protein stream originating from step (a)-(iii) and a lactose source to obtain a recombined stream;
(b2) pasteurizing the whey protein stream originating from step (b);
(c) using the stream originating from step (b2) in the manufacture of the nutritional composition;
wherein step (a)(i) involves subjecting the defatted milk to microfiltration over a membrane capable of retaining bacteria and permeating milk proteins;
or,
(a) processing defatted milk into a casein stream, a whey protein stream and a lactose stream, by:
   (i) subjecting the defatted milk to a debacterialization treatment, to provide a debacterialized milk;
   (ii) subjecting the debacterialized milk originating from step (i) to cold microfiltration over a membrane capable of retaining casein and permeating whey proteins, to provide a casein stream as retentate and a permeate comprising whey protein and β-casein;
   (iii) fractionating the permeate originating from step (ii) into a whey protein stream comprising 1) whey protein and β-casein and 2) a lactose stream;
(b) combining at least part of the whey protein stream originating from step (a)-(iii) and a lactose source to obtain a recombined stream;
(c) using the stream originating from step (b) in the manufacture of the nutritional composition;
wherein step (a) (i) involves pasteurizing the defatted milk.

### DETAILED DESCRIPTION

The inventors surprisingly found that the native whey protein composition according to the invention significantly improved tolerance in the gastrointestinal tract. The native whey protein composition is preferably comprised in a nutritional composition, more preferably a preterm formula, an infant formula or follow-on formula.

### Native Whey Protein Composition

The whey protein composition according to the invention comprises whey protein with an increased nativity compared to whey proteins that are typically comprised in nutritional compositions. The inventors have found that partially or fully replacing the conventional whey protein fraction in such a nutritional composition with the native whey protein according to the invention provides a beneficial effect in relation to gastrointestinal tolerance. The present invention does not concern breastfeeding, and the native whey protein (composition) in the context of the invention is non-human whey protein (composition). Although any whey protein is suitable in the context of the present invention, the whey protein is preferably bovine whey protein.

Native whey proteins comprise mainly α-lactalbumin, and β-lactoglobulin. Native whey protein is directly extracted/removed from milk, preferably unpasteurized milk, with the use of cold microfiltration or ultrafiltration. Native whey protein according to the invention has not been subjected to the cheese making process, and is therefore free from remnants of rennet, lactic acid bacteria, bacteriophages, somatic cells, cheese fines, and glycomacropeptide. Native whey proteins are substantially undenatured and have not been exposed to extensive heat treatment or pressure.

The native whey protein according to the invention is subjected to pasteurization, preferably mild pasteurization, at a temperature above 63° C., preferably a least 70° C. Pasteurization is known in the art and may e.g. involve HTST, ESL or UHT. The pasteurization step as meant herein is a mild pasteurization step and has the purpose of reducing the microbial load to such an extent that the resulting nutritional composition is substantially free from live microorganisms and safe for consumption, even by infants. In particular, it is safe with regards to Bacillus cereus and Enterobacter sakazalth, for instance, such as laid down in European Regulation No 2073/2005 dated 2007, corrigendum No. 1441/2007. The mild pasteurization conditions are selected to result in the desired reduction in bacterial load while preserving the whey proteins nativity, preferably resulting in whey proteins having a nativity of more than 80%, preferably more than 90%, more preferably as described here above. Preferably, mild pasteurization as applied in the context of the invention preferably involves heating at 72-74° C. for 15 to 30 seconds. However, it is considered to fall within the skilled person's ambit to establish a corresponding combination of time and temperature to arrive at a pasteurized composition with the appropriate relative amount of native whey proteins.

The native whey protein or the composition it is comprised in is thus pasteurized. The inventors of the present invention have surprisingly shown that the native whey protein, obtained according to the present invention that includes a pasteurization step, can be used for the treatment and/or prevention of gastrointestinal intolerance. Pasteurized compositions are substantially free of alkaline phosphatase activity. Thus, in a preferred embodiment, the native whey protein or the composition it is comprised in is a composition, preferably an infant formula, is substantially free of alkaline phosphatase activity. In a preferred embodiment, the term substantially free of alkaline phosphatase activity means that, when measured using a liquid composition, the alkaline phosphatase activity is below 350 mU/L. In a preferred embodiment, the invention relates to an infant formula comprising the native whey protein for use in the treatment and/or prevention of gastrointestinal intolerance. The infant formula product is preferably pasteurized and substantially free of alkaline phosphatase activity. Alternatively worded, the native whey protein or the composition it is comprised in has a low bacterial load, as is obtainable by a pasteurization step. Preferably, the composition has no detectable amounts of Listeria monocytogenes, Salmonella and/or Enterobacter sakazakil Herein, the levels of Listeria monocytogenes are typically applicable to liquid ready-to-feed infant formulae, and can be determined by EN/ISO 11290-1, and the levels of Salmonella and Enterobacter sakazakil are typically applicable to dried infant formulae, and can be determined by EN ISO 6579-1 and EN ISO 22964 respectively.

The increased nativity of the whey protein according to the invention can be defined in terms of a nativity of more than 80%, preferably more than 90%, more preferably more than 92%, preferably more than 94%, preferably more than 95%, even more than 98%. Hence, in the context of the invention, the whey protein composition of the invention comprises whey protein of which more than 80%, preferably more than 90%, more preferably more than 92%, preferably more than 94%, preferably more than 95%, even more than 98% is native whey protein; non-native whey protein makes up for the remainder of the whey protein, to obtain a total of 100% whey protein. Nativity is a known parameter in the art and can be determined by any means available to the skilled person. The nativity refers to the percentage of native protein of a particular type based on the total amount of protein of the same type. Here, the nativity of the whey protein refers to the amount of native whey protein based on the total amount of whey protein. In one embodiment, the nativity is determined according to a suitable Kjeldahl-based analysis, such as per the ISO 8968-3/IDF 20-3:2004. Hence, it is preferred that the above ranges of nativity are determined according to ISO 8968-3/IDF 20-3:2004, or as described in Delahaije, R. J Journal of agricultural and food chemistry 2016, 64, 4362-4370, herewith incorporated by reference. As described therein, non-native proteins are precipitated by adjusted the pH of the protein composition to 4.6 with 0.1 M HCl, and the protein content may then be determined by ISO 8968-3/IDF 20-3:2004. Further details are provided in the examples.

In one embodiment, the nativity of the whey protein in the composition is in the range of 90-100%, preferably in the range of 92-99%, more preferably in the range of 94%- [symbol] % of the whey [symbol] . Being the two most abundant whey proteins, it is especially preferred that - [symbol] and -lactoglobulin have high nativity. The inventors surprisingly found that especially - [symbol] remained largely native in the process according to the present invention. It is thus preferred that -lactalbumin has a nativity of it [symbol] least 70%, more preferably 75-95%, most preferably 78-85%. Likewise, it is preferred that -lactoglobulin has a nativity of at least 70%, more preferably 80-100% % [symbol] preferably 85- [symbol] %. Without wishing to be [symbol] by any theory, it is believed that the nativity of -lactalbumin and/or -lactoglobulin, especially -lactoglobulin, contribute to the beneficial effects on gastrointestinal tolerance.

The native whey protein composition according to the invention comprises native whey protein and may comprises further proteins, but preferably the amount of proteins other than native whey protein which is subjected to the mild heat treatment according to the invention is not more than 40 wt %, preferably not more than 35 wt %, more preferably at most 30 wt %, most preferably 15-30 wt %, based on total protein in the heat-treated composition. The protein other than native (and small amounts of non-native) whey protein in the native whey protein composition is preferably beta-casein. Thus, in one embodiment, the casein content of the native whey protein composition according to the invention is at most 45 wt %, more preferably at most 40 wt %, more preferably at most 35 wt %, even more preferably at most 30 wt %, even at most 28 wt %, based on total protein. In one embodiment the casein content of the native whey protein composition according to the invention consists of beta-casein. In a preferred embodiment the ratio of whey protein with a nativity of at least 80%, more preferably as defined according to one of the preferred embodiments described herein, to beta-casein in the native whey protein composition is between 80:20 and 55:45 more preferably between 80:20 and 70:30. In an embodiment, the native whey protein composition may also comprises free amino acids, preferably in amounts up to 5%, more preferably up to 2% of all proteinaceous matter.

The native whey protein composition according to the invention is substantially devoid of alpha-casein and kappa-casein, preferably comprising less than 6 wt %, preferably less than 4 wt %, more preferably less than 2 wt % of the sum of alpha-casein and kappa-casein, based on the total protein weight of the native whey protein composition. In an embodiment the native whey protein composition does not comprise any detectable amounts of alpha-casein and kappa-casein. In an embodiment the native whey protein composition does not comprise alpha-casein and kappa-casein.

The native whey protein composition according to the invention typically comprises beta-casein. In a preferred embodiment the weight ratio of beta-casein to the sum of alpha-casein and kappa-casein in the native whey protein composition is preferably in a range between 99:1 and 90:10, more preferably between 99:1 and 95:15. In a preferred embodiment between 90%-100% of the casein in the native whey protein composition is beta-casein, more preferably 95%-99% of the casein is beta-casein. In one embodiment 100% of the casein in the native whey protein composition is beta-casein. In case the desired protein composition comprises both casein and whey proteins, such as for many dairy products in particular infant formula products, the inventors found that beta-casein gave less protein coagulation, whereas the presence of alpha-casein led to increased protein coagulation. Thus, beta-casein in combination with native whey provided unexpected good effects in the treatment and/or prevention of gastrointestinal intolerance.

The native whey protein composition according to the invention has been heat-treated. In a preferred embodiment the native whey and native whey protein composition have been pasteurized. Pasteurization of the native whey protein is preferably performed at 72-74° C. for 15 to 30 seconds.

The native whey protein composition according to the invention has a protein solubility of more than 55%, preferably more than 60%, more preferably more than 65%, more than 68%, more than 70% or even more than 71% based on the total amount of protein in the native whey protein composition at acidic pH conditions, preferably at a pH of 4.5-4.7. The protein solubility is a known parameter in the art and can be determined by any means available to the skilled person. It is preferred to determine the above solubility as described in example 2, making use of the fact that native whey protein is soluble at pH 4.5-4.7 whereas beta-casein is not soluble at the same pH range.

Additionally or alternatively, the native whey protein composition according to the invention is obtainable by membrane-filtration based technology, preferably cold membrane filtration based technology.

In a preferred embodiment, the native whey does not originate from acid whey or from sweet whey. In other words, the whey has not undergone a step wherein casein is precipitated.

It is furthermore preferred that the whey has not undergone a treatment step during manufacturing wherein the pH is lowered to a value below 6, preferably not below 5.5.

A preferred process for preparing the native whey protein composition, or the nutritional composition comprising the native whey protein is referred herein as the process according to the invention and is further defined below.

### Nutritional Composition

The native whey protein according to the invention is preferably comprised in a composition, such as a nutritional composition. The composition according to the invention, further defined here below, is ideally suitable for reducing and/or preventing the occurrence of gastrointestinal intolerance according to the present invention. In one embodiment, the composition according to the invention is obtainable by or obtained by the process according to the invention as further defined below. In one embodiment, the native whey protein composition is provided in a packaged and sealed form. The nutritional composition according to the invention comprises a protein fraction comprising the native whey protein according to the invention, and typically one or more of, most preferably all of, a lipid fraction, a digestible carbohydrate fraction, dietary fibres, vitamins and minerals. Preferably, the nutritional composition according to the invention is a complete nutrition, meaning that it comprises at least protein, lipids and digestible carbohydrates.

The native whey protein according to the invention is preferably comprised in a nutritional composition, more preferably in an infant formula, most preferably a preterm formula. Preferably, the whey protein fraction of the nutritional composition contains at least 80 wt %, preferably at least 90 wt %, of whey proteins being the native whey proteins as defined herein. Most preferably, the nutritional composition is substantially free of whey proteins other than the native whey proteins as defined herein. The nutritional composition according to a preferred embodiment of the invention is substantially devoid of alpha-casein and kappa-casein, preferably comprising less than 6 wt %, preferably less than 4 wt %, more preferably less than 2 wt % of the sum of alpha-casein and kappa-casein, based on the total protein weight of the protein fraction. The nutritional composition according to the invention is preferably for oral feeding, as that provides the least impact on regular feeding regimes. Nonetheless, the nutritional composition comprising native whey proteins in the form of a tube feed or other feed is also suitable for reducing and/or preventing gastrointestinal intolerance.

In the context of the present invention, “infant formula” refers to milk-based nutritional compositions suitable for feeding infants, which typically are in the form of a reconstitutable powder or a ready-to-feed liquid composition. Preferably, the composition is an infant formula, a follow-on Formula, a growing-up milk, or a base therefore. The terms ‘infant formula’ and ‘follow-on formula’ are well-defined and controlled internationally and consistently by regulatory bodies. In particular, CODEX STAN 73-1981 “Standard For Infant Formula and Formulas For Special Medical Purposes Intended for Infants” is widely accepted. It recommends for nutritional value and formula composition, which require the prepared milk to contain per 100 ml not less than 60 kcal (250 kJ) and no more than 70 kcal (295 kJ) of energy. FDA and other regulatory bodies have set nutrient requirements in accordance therewith. Most preferably, the composition is an infant formula. The infant formula may be a powder, preferably a spray-dried powder, intended to be reconstituted into a liquid infant formula, or a liquid infant formula.

An especially preferred infant formula in the context of the present invention is a preterm infant formula. The protein intake recommendation of preterm infants with a birth weight of less than 1000 g is 4.0-4.5 gkg·-1d-1. Accordingly, the preterm formula of the present invention may comprise a protein concentration corresponding to a protein intake of 4.0-4.5 g·kg-1d-1, suitably 4.1-4.5 g·kg-1d-1, more suitably 4.2-4.4 g·kg-1d-1. The preterm formula may comprise 3.2-4.1 g protein per 100 kcal, suitably 3.6-4.1 g protein per 100 kcal, more suitably 3.7-4.1 g protein per 100 kcal. Suitably, such preterm formula is intended for a preterm infant weighing <1000 g at birth.

In one embodiment, the nutritional composition comprises native whey protein obtainable by step (a) of the process as detailed further below. More preferably, the nutritional composition is obtainable by step (a) of the process as detailed further below. Thus, in one embodiment, the composition according to the present invention comprises a whey protein fraction which is obtainable as a whey protein stream via the process of the present invention as defined below, in particular step (a). In particular, the whey proteins are obtainable by subjecting a defatted, debacterialized milk to microfiltration over a membrane capable of retaining casein and permeating whey proteins to provide a permeate comprising lactose, whey protein and beta-casein and fractionating the permeate using ultrafiltration into a whey protein stream comprising 1) whey protein and beta-casein and 2) a lactose stream, wherein debacterialization is preferably performed by microfiltration or pasteurization. The whey proteins and beta-casein are present in the thus obtained ultrafiltration retentate. In one embodiment, the whey protein is obtained by the process defined herein. It is well-known to the skilled person how to obtain such an ultrafiltration retentate that contains native whey proteins starting from defatted milk.

In case the nutritional composition is an infant formula or follow-on formula, it is typically nutritionally complete for infants, and contains all necessary macronutrients and micronutrients for infant formulas as known in the art. Specifically, the formula preferably contains casein, in addition to the native and intact whey protein.

In an embodiment, the formula preferably comprises, calculated by weight of total casein, 80-100 wt % beta-casein, more preferably 85-100 wt. % beta-casein, more preferably 95-100 wt. % beta-casein provided by the native whey composition. In other words, the formula is preferably substantially devoid of alpha-casein and kappa-casein, preferably comprising less than 6 wt %, preferably less than 4 wt %, more preferably less than 2 wt % of the sum of alpha-casein and kappa-casein, based on the total protein weight of the protein fraction. In view of the absence of alpha-casein and kappa-casein, the formula preferably comprises lysine in the range of 60-90 mg/g protein, more preferably in the range of 65-85 mg/g protein. As such, the protein composition better reflects the protein composition of human milk.

In an embodiment the native whey protein composition is the sole protein source of the formula which may however optionally be supplemented with free amino acids, preferably less than 2 wt % of all proteinaceous matter. The preferred free amino acids are tyrosine and cysteine, preferably at least tyrosine, in an amount from 0.1 to 2 wt % based on the total weight of protein in the formula. Depending on the amount of protein in the formula, these amino acids may be supplemented to improve the amino acid profile in accordance with amino acid requirements for infant formulas.

In one embodiment, the nutritional composition according to the invention shows a negative reaction to an alkaline phosphatase (ALP) activity test. As the skilled person will understand, this is typically achieved by a pasteurization step as defined herein. Tests for alkaline phosphate activity are known in the art and are used as standard for defining the activity (or lack of activity) of the enzymes in an infant formula. The law, for example European Regulation 2074/05, and its amendment in EC No 1664/2006, requires the ALP activity to be below 350 mU/L, which is also referred to as ALP negative. The ALP activity can be defined as mU/g (typically for powders, or for liquids based on dry weight) or mU/L (typically for liquids, including reconstituted powders). In case the nutritional composition is a preterm formula, it is preferred that the nutritional composition is ALP negative. Thus, in one embodiment, the ALP activity of the composition according to the invention, when in liquid form, is below 350 mU/L, or, when in powder form, is typically below 350 mU/L after reconstitution as common in the art of infant formulas and follow-on formulas. In one embodiment, the ALP activity is determined by ISO standard 11816-1.

### The Process for Making Native Whey Protein or the Nutritional Composition

In one embodiment, the native whey protein composition is obtainable by membrane-filtration based technology in order to retain the nativity of the whey proteins. Preferably, the process for obtaining the native whey protein composition comprises:
(a) processing defatted milk into a casein stream, a whey protein stream and a lactose stream, by:
   (i) subjecting the defatted milk to a debacterialization treatment, to provide a debacterialized milk;
   (ii) subjecting the debacterialized milk originating from step (i) to cold microfiltration over a membrane capable of retaining casein and permeating whey proteins, to provide a casein stream as retentate and a permeate comprising whey protein and β-casein;
   (iii) fractionating the permeate originating from step (ii) into a whey protein stream comprising 1) whey protein and β-casein and 2) a lactose stream;
(b″) optionally spray-drying the whey protein stream originating from (a)-(iii) followed by dissolving;
   wherein at least one of the debacterialization treatment of step (a)-(i) or the stream originating from step (a)-(iii) after optional step (b″) is subjected to pasteurization according to the invention; and;
(c″) optionally freeze-drying the stream,
   and wherein the debacterialization treatment (i) if not pasteurization, involves subjecting the defatted milk to microfiltration over a membrane capable of retaining bacteria, wherein the debacterialized milk is in the permeate.
   The pasteurization treatment according to the invention preferably involves mild pasteurization.

In one embodiment, the nutritional composition is obtainable by the process comprising the aforementioned steps (a)-(i), (a)-(ii) and (a)-(iii), and
(b) combining at least part of the whey protein stream originating from step (a)-(iii) and a lactose source to obtain a recombined stream;
(c) using the recombined stream originating from step (b) in the manufacture of the nutritional composition,
   wherein at least one of the debacterialization treatment of step (a)-(i) or the recombined stream originating from (b) is subjected to pasteurization according to the invention; and wherein the debacterialization treatment (a)-(i) if not pasteurization, involves subjecting the defatted milk to microfiltration over a membrane capable of retaining bacteria, wherein the debacterialized milk is in the permeate. In a preferred embodiment the recombined stream does not comprise the casein stream or parts thereof.

In one embodiment, the nutritional composition is obtainable by the process comprising: (a) processing defatted milk into a casein stream, a whey protein stream and a lactose stream, by the aforementioned steps (a)-(i), (a)-(ii) and (a)-(iii),

wherein the debacterialization treatment (a)-(i) involves subjecting the defatted milk to microfiltration over a membrane capable of retaining bacteria, wherein the debacterialized milk is in the permeate,
wherein fractioning (a)-(iii) involves subjecting the permeate originating from (a)-(ii) to ultrafiltration;
(iv) optionally spray drying the whey protein stream originating from step (a)-(iii) followed by dissolving;
(b) combining at least part of the whey protein stream originating from step (a) and a lactose source to obtain a recombined stream;
(b2) mild pasteurization of the stream originating from step (b),
(c) using the stream originating from step (b2) in the manufacture of the nutritional composition.
   The nutritional composition obtainable by the process does not include combining the casein stream or part thereof originating from step (a) with the at least part of the whey protein stream originating from step (a) and a lactose source to obtain a recombined stream of step (b).

In the process according to the invention defatted milk is treated to produce a nutritional composition. In the context of the present invention, whenever a certain stream or composition is mentioned to “originate from” a certain process step, such as from the recombined stream originating from step (b), said stream or composition can be the composition which is directly obtained by said process step. In addition, if such a directly obtained stream or composition undergoes one or more additional processing steps, such as partial evaporation and/or supplementation of additional water or other components, the stream or composition is also regarded to originate from that specific process step. Thus, if the stream of step (b) would be partially evaporated, the incoming stream of step (c) is still regarded to be the recombined stream originating from step (b). In the context of the present invention, the term “stream” refers to a liquid composition, although the presence of some solid material is not excluded, e.g. as in a suspension, as long as the composition can be handled by conventional dairy plants.

The present process uses milk as starting material in step (a). Defatted milk, preferably defatted cow's milk, is subjected to step (a). In the context of the invention, “defatted milk” refers to milk having a reduced fat content compared to whole milk. Typically, the fat content of the defatted milk is in the range of 0-2 wt %, preferably 0-1 wt %, more preferably 0-0.2 wt %, most preferably 0-0.05 wt %, based on total weight of the defatted milk. In one embodiment, the defatted milk is skim milk. The present process employs milk, which refers to non-human milk, preferably cow's milk. Most preferably, cow's skim milk is used. In one embodiment, the process comprises a step of defatting milk to obtain the defatted milk, which is subsequently subjected to step (a). Herein, non-defatted milk, or just milk or whole milk, is subjected to the defatting step. The defatting step affords the defatted milk. Preferably, the defatted milk is the sole protein source for the nutritional composition.

### Step (a)

In step (a), the defatted milk is processed or fractioned into a casein stream, a whey protein stream and a lactose stream. Herein, the casein stream is a liquid composition comprising casein, which is enriched in casein compared to the casein content in the incoming defatted milk, the whey protein stream is a liquid composition comprising whey protein, which is enriched in whey protein compared to the whey protein content in the incoming defatted milk and the lactose stream is a liquid composition comprising lactose, which is enriched in lactose compared to the lactose content in the incoming defatted milk. In the context of the present invention, “enriched” is defined that the content of the enriched component, based on dry weight, is increased in one stream compared to another stream. Thus, the casein stream is enriched in casein, i.e. has a higher casein content, based on dry matter, compared to the incoming defatted milk.

The fractionation of step (a) is preferably accomplished by membrane filtration techniques and involves a combination of microfiltration and ultrafiltration. The casein stream originates from the microfiltration as retentate, the whey protein stream originates from the ultrafiltration as retentate and the lactose stream originates from the ultrafiltration as permeate. Suitable membrane filtration processes are known in the art, e.g. as disclosed in WO 2013/068653, WO 2013/137714 and WO 2015/041529. More specifically, step (a) includes:
(i) subjecting the defatted milk to microfiltration over a membrane capable of retaining bacteria and permeating milk proteins, to provide a debacterialized milk;
(ii) subjecting the permeate originating from step (i) to microfiltration over a membrane capable of retaining casein and permeating whey proteins, to provide a casein stream as retentate and a permeate comprising whey protein; and
(iii) fractionating the permeate originating from step (ii) into a whey protein stream and a lactose stream.
   More specifically step (ii) includes subjecting the permeate originating from step (i) to cold microfiltration over a membrane capable of retaining casein and permeating whey proteins, to provide a retentate comprising casein and a permeate comprising whey protein and beta-casein.

The incoming defatted milk is subjected to debacterialization (bacterial removal) in step (i). Debacterialization may be performed by filtration or by pasteurization. In one embodiment, debacterialization is performed by bacterial filtration (e.g. microfiltration (MF)). Such filtration processes to reduce the bacterial load of milk are known in the art. The microfiltration of step (i) may be performed by microfiltration over a membrane capable of retaining bacteria and permeating milk proteins, to provide a debacterialized milk as permeate. Preferably, the microfiltration of step (i) [symbol] ceramic microfiltration. The [symbol] membrane preferably has a pore size of between 1.8 and m, preferably between 1.4 and 0.8 m. The MF process of step (i) is preferably executed at a temperature of between 4 and 20° C., more preferably between 8 and 15° C., most preferably at a temperature of about 10° C.

Alternatively, step (i) is performed by pasteurization. Pasteurization and preferred embodiments thereof are described in more detail below in the context of step (b2), which equally applies here.

In the microfiltration step (ii), the debacterialized milk originating from step (i) is fractioned into two distinct streams, each enriched in a particular protein type; a casein enriched MF retentate (MFR) and a whey protein enriched MF permeate (MFP) are produced. The MF step (ii) is performed over a membrane that enables [symbol] of casein and whey proteins. Such [symbol] membrane typically has a porosity of between 0.05 and 0.5 m, more preferably between 0.08-0.35 m. Alternatively, the membrane used in step (ii) may have a molecular weight cut-off in the range of 250-1500 kDa, preferably in the range of 500-1000 kDa. Preferably, a ceramic membrane or a spiral wound (organic) membrane is used. Microfiltration of step (ii) is preferably performed with a volume concentration factor (VCF) in the range of 1.5-10, preferably 2-5, which has been found to provide the most optimal results in terms of the composition of the MF retentate, especially in terms in terms of casein content. Preferably the microfiltration step (ii) is a cold microfiltration step wherein the MF step (ii) is preferably performed at a temperature of between 4 and 15° C., more preferably between 6 and 12° C., most preferably at a temperature of between 8 and 10° C. Beneficially, cold microfiltration typically results in a retentate comprising casein and a permeate comprising whey protein and β-casein.

In the context of the invention, the term “volume concentration factor” or “VCF” is the factor at which a liquid composition is concentrated upon filtration, i.e. the total volume of the incoming stream prior to filtration divided by the total volume of the retentate after filtration, irrespective of the total solid content. Thus, when 5 L of a liquid composition is fractionated over an ultrafiltration membrane into a permeate of 4 L and a retentate of 1 L, this UF process operates with a VCF of 5/1=5.

According to a preferred embodiment, microfiltration of step (ii) is enhanced with diafiltration (DF). Diafiltration may be accomplished by diluting the retentate of the MF at least once with an amount of water, or by diluting the incoming debacterialized milk with an amount of water and subjecting the diluted milk to MF. The DF water may be added to the incoming debacterialized milk or MFR at once, or the total amount of DF water may be added in several fractions. After each addition of DF water to the incoming skim milk or MFR, the diluted liquid composition is subjected to MF.

Fractionation of a composition comprising whey protein and lactose into a composition enriched in whey protein and a composition enriched in lactose is known in the art. Step (iii) is preferably performed by ultrafiltration (UF). During ultrafiltration, most of the liquid and small solutes end up in the UF permeate (UFP), while the UF retentate (UFR) comprises substantially all whey protein, in a smaller volume. Small molecules which permeate through the UF membrane are for example lactose, monovalent and polyvalent ions. The ultrafiltration of step (iii) can be carried out with any UF membrane known in the art, including ceramic membranes, tubular and organic spiral wound membranes. Preferably the UF membrane is an organic spiral wound membrane. The UF membrane has a molecular weight cut-off of that enables proteins, preferably whey proteins, to remain in the retentate, and allow small solutes, for example lactose, to permeate through the membrane. The UF step (iii) preferably is carried out with a membrane having a molecular weight cut-off of at most 25 kDa, more preferably at most 10 kDa, and preferably of at least 2.5 kDa, more preferably at least 5 kDa. The UF step (iii) is preferably carried out with a volume concentration factor (VCF) in the range of 20-200, preferably 50-150, which has been found to provide the most optimal results in terms of the composition of the UF retentate.

Step (a) may further comprise one or more concentration steps, such as concentration of the MFR originating form step (ii) and/or the UFR originating form step (iii). Concentration is preferably performed by reverse osmosis (RO), nanofiltration (NF) and/or evaporation. NF is most preferred, as NF concentrates the stream and at the same time lowers the monovalent ion content, which are able to permeate the NF membrane. Such lowering of the monovalent ion content is especially desirable in the production of infant formulas.

The protein fraction of the casein stream originating from step (a) typically comprises very little whey protein, preferably less than 15 wt %, more preferably less than 10 wt %, based on the weight of the protein fraction of the casein stream, and is high in casein. Preferably the protein fraction comprises at least 85 wt % casein, more preferably at least 90 wt % casein. The content of total solids in the casein stream typically ranges from 5 to 30 wt %, preferably from 7 to 30 wt %, most preferably from 17 to 24 wt %, based on total weight of the casein stream. The casein stream may also be referred to as a casein concentrate, casein isolate, micellar casein concentrate or micellar casein isolate (MCI).

The whey protein stream is typically a liquid composition having a total solid content of 5-35 wt %, preferably of 10-30 wt %, most preferably of 20-30 wt %, and typically comprises 25-90 wt %, preferably 60-85 wt % whey proteins based on total dry weight. The whey protein stream may also be referred to as an aqueous composition comprising whey proteins. Although the whey protein stream is enriched in whey protein compared to the incoming defatted milk, it may still contain substantial amounts of casein, depending on the exact conditions at which the fractionation between casein and whey protein by ultrafiltration, is performed. In one embodiment, the whey protein stream comprises at most 45 wt %, preferably at most 40 wt %, more preferably at most 30 wt %, preferably 15-30 wt %, more preferably 20-30 wt % beta-casein based on total weight of the protein. Variations in the fractionation conditions and the accompanying changes in the whey protein stream are known in the art. In one embodiment the whey protein stream is obtained with cold microfiltration and typically comprises whey protein and β-casein in a ratio between 85:15 and 55:45, preferably between 80:20 and 55:45, more preferably between 80:20 and 60:40, more preferably between 80:20 and 70:30.

The lactose stream is typically a liquid composition having a total solid content of 3-30 wt %, preferably of 5-22 wt %. The lactose content in the lactose stream originating from step (a) is typically at least 75 wt %, preferably at least 90 wt % or even at least 95 wt %, based on total dry weight.

### Demineralization

The process according to the invention preferably comprises a demineralization step, wherein the lactose source, or one or more components thereof, is/are demineralized prior to being subjected to step (b) wherein at least part of the whey protein stream may be combined with a lactose source to obtain a recombined stream. Demineralization is thus typically performed on at least part of the lactose stream originating from step (a) prior to being subjected to step (b). Demineralization is particularly preferred for the manufacture of infant formulas, for which it is typically required to lower the mineral content as compared to the incoming milk. Thus, in one embodiment, at least part of the lactose stream originating from step (a), preferably the UFP originating from step (iii), is subjected to demineralization prior to being used as (part of) the lactose source in step (b).

Demineralization of the lactose source may be performed by any technique known in the art, such as electrodialysis, ion exchange, salt precipitation, lactose crystallization, membrane filtration techniques such as nanofiltration, optionally enhanced with diafiltration, or combinations thereof. In a preferred embodiment, demineralization comprises at least one of salt precipitation, electrodialysis, lactose crystallization and ion exchange, optionally in combination with nanofiltration, more preferably demineralization comprises nanofiltration in combination with at least one of salt precipitation, electrodialysis, lactose crystallization and ion exchange. In preferred embodiment, demineralization comprises at least electrodialysis and/or salt precipitation. In one preferred embodiment, demineralization comprises at least nanofiltration in combination with electrodialysis and/or salt precipitation. The inventors found that when only nanofiltration is used for demineralization, especially for demineralization of an ultrafiltration permeate as lactose source in the preparation of infant formulas and follow-on formulas, the content of divalent ions, such as calcium and phosphate, is typically insufficiently reduced to obtain a final infant formula or follow-on formula within legal requirement.

Demineralization is preferably performed such that at least 20 wt %, or preferably 50 wt %, more preferably at least 70 wt % or at least 80 wt %, most preferably at least 90 wt % of the polyvalent ions and/or such that at least 20 wt % of the monovalent ions are removed, more preferably at least 35 wt % or at least 50 wt %, most preferably at least 60 wt % of the monovalent ions, present in the lactose stream, e.g.t the UFP originating from step (iii), are removed.

### Step (b)

In step (b), at least part of the whey protein stream originating from step (a) and a lactose source are combined to obtain a recombined stream. This recombined stream is used to manufacture the nutritional composition in step (c), after a pasteurization step (b2). Step (b) thus does not include combining the casein stream or part thereof originating from step (a) with the at least part of the whey protein stream originating from step (a) and a lactose source to obtain a recombined stream of step (b). It is part of the gist of the invention to proceed with little casein other than beta-casein. The combining of step (b) may involve additional components. The whey protein to beta-casein weight ratio in the recombined stream is in the range between 85:15 and 55:45, preferably between 80:20 and 55:45, more preferably between 80:20 and 60:40, more preferably between 80:20 and 70:30. In one embodiment, the whey protein to casein weight ratio in the recombined stream is about 72:28. The exact ratio of whey protein to beta-casein is typically determined by the sieving coefficient of the cold microfiltration step a-ii.

Preferably, all of the whey protein stream originating from step (a) is subjected to the combining of step (b). In one embodiment, 0-50 wt %, preferably 5-25 wt %, based on total weight of the lactose, of the lactose stream originating from step (a) is subjected to step (b) as (part of) the lactose source. The amount of the lactose stream originating from step (a) that is subjected to step (b) as (part of) the lactose source is advantageously governed by the amount of lactose required for step (d). In case the amount of lactose in the lactose stream originating from step (a) that is subjected to step (b) would be insufficient for infant formula manufacture, additional lactose can be used. In one embodiment, all of the whey protein stream and part of the lactose stream are combined. In one embodiment, all of the whey protein stream and all of the lactose stream are combined. In one embodiment, all of the whey protein stream and nothing of the lactose stream are combined. In a preferred embodiment all of the whey protein stream is combined with part or all of the lactose stream and nothing of the casein stream. In one embodiment, at least part of the UFR originating from step (iii) and at least part of the UFP originating from step (iii) are combined.

In step (b), two or more streams are recombined into one stream. This recombining may occur at once (streams are combined simultaneously) or step-wise (streams are combined consecutively). Combining can be performed as wet mixing or as dry mixing or even as a combination of both.

Preferably, the combining occurs as wet mixing, wherein liquid compositions are mixed in the appropriate amounts.

Step (b″) is applied in the process for obtaining the native whey protein composition, whereas step (b) is applied in the process for obtaining a nutritional composition. In step (b″) the whey protein stream originating from step (iii) is optionally spray-dried followed by dissolving by conventional means known in the art.

Pasteurization According to the Invention, in Step (a)-(i) or (b2)

The process according to the invention contains a pasteurization step. The pasteurization step may be performed as step (a)-(i) or as step (b2). A pasteurization step is a requirement for infant formulas in many jurisdictions from a food safety perspective. In a preferred embodiment, the process of the invention contains only a single pasteurization step to ensure the obtained product is sufficiently heat-treated with regards to prevention of microbial or bacterial contaminations but on the other hand ensures preservation of protein nativity. In a preferred embodiment, the process of the invention contains a mild pasteurization step. In a case the composition is subjected to multiple pasteurization steps and/or also sterilization steps, it is preferred that at least the first heat treatment which is applied to the composition is the pasteurization as detailed here above.

In a preferred embodiment, step (i) is a filtration step and step (b2) is performed. Although the incoming defatted milk may be pasteurized in step (i), it is preferred that if a pasteurization step is included, the recombined stream originating from step (b) is subjected to a pasteurization step (b2) prior to being subjected to step (c).

Pasteurization is described here above.

### Step (c)

In step (c), the recombined stream originating from step (b) or (b2) is used to manufacture the nutritional composition. Such manufacturing is known in the art and typically involves one or more of drying, concentrating, supplementing with vitamins, minerals, lipids and/or dietary fibres, heat treatment, homogenisation, packaging. In a preferred embodiment, step (c) does not involve heat treatment, and involves one or more of drying, concentrating, supplementing with vitamins, minerals, lipids and/or dietary fibres and packaging. Preferably, step (d) involves at least a drying step, most preferably it involves all of the above mentioned steps. In a preferred embodiment, a drying step is performed directly after step (b) or (b2), most preferably directly after step (b2).

Although one or more of the separate streams may be dried prior to being combined in step (b), it is preferred that the recombined stream originating from step (b) is dried, preferably spray-dried. As such, only one drying step is needed in the manufacture of the nutritional composition. In a preferred embodiment, the process according to the invention comprises only a single drying step, wherein in step (c) the recombined stream is dried, preferably by spray-drying. Due to the inherently limited heat-load as a consequence of low water activity of droplets produced during spray-drying, protein nativity remains substantially the same and is not significantly impacted during spray-drying. This allows that the content of native protein in the final nutritional composition is as high as possible and substantially the same as prior to spray-drying. To retain the native protein content in the final product, the spray-drying step is preferably executed with an inlet temperature of less than 250° C., preferably less than 220° C., more preferably less than 200° C. Alternatively worded, the spray-drying step is executed such that the wet bulb temperature is kept below 80° C., preferably below 70° C. or even below 50° C. Using such spray-drying conditions, nativity of the proteins that are spray-dried will not be impacted anymore due to the low water activity of the powder particles in the spray-drier. In one embodiment, the recombined stream is concentrated, preferably prior to being dried. Such concentration may be accomplished by any means known in the art, such as by reverse osmosis (RO), nanofiltration (NF) and/or evaporation.

Depending on the desired type of nutritional composition, supplementation of certain components, such as amino acids, vitamins, minerals, lipids and/or dietary fibres, may be desired. Such supplementation can be performed either prior to, during or after combining step (b) and/or optionally prior to or after a drying step. The skilled person is aware of the requirements of particular types of nutritional compositions, especially infant formulas, e.g. from EU directive 91/321/EEC or EU directive 2006/141/EC or US Food and Drug Administration 21 CFR Ch 1 part 107, and is able to adjust the composition of the recombined stream in order to meet those requirements.

As will be appreciated by the skilled person, process steps that lead to denaturation of the whey protein should be avoided as much as possible. For example, the nutritional composition may be a spray-dried powder, in which case it is preferred that that the spray-drying step is executed with an inlet temperature of less than 250° C., preferably less than 220° C., more preferably less than 200° C. It is preferred that the whey proteins have undergone a pasteurization step preferably a single pasteurization step.

In one aspect, the present invention concerns the intact whey protein composition as present in the whey protein stream obtainable by the process according to the invention, i.e. obtainable by step (a), which is comprised by an infant formula product obtainable by step (c). Said whey protein stream exhibits improved gastric coagulation behaviour. Said intact whey protein stream preferably comprises intact whey protein and beta-casein in a ratio of whey protein:casein in the range of 85:15 and 55:45, preferably between 80:20 and 55:45, more preferably between 80:20 and 60:40, more preferably between 80:20 and 70:30. The intact whey protein composition obtainable by the present invention exhibits upper gastrointestinal coagulation kinetics closer to human milk than currently commercialized infant formula.

The skilled person is capable of determining the extent of gastric protein coagulation, e.g. from Van den Braak et al. (Clin. Nutr. 2013, 32, 765-771). A preferred method for determining gastric coagulation is according to the semi-dynamic digestion model of Example 8.

It is noted that step (c″) is applied (instead of step (c)) in the process for obtaining a native whey protein composition according to the invention, and step (c) is applied in the process for obtaining a nutritional composition according to the invention. In step (c″) the stream originating from step (b″) may optionally be freeze-dried by conventional means known in the art.

### Application

The inventors found that the native whey protein composition subjected to mild pasteurization treatment according to the invention is capable of reducing and/or preventing the occurrence of gastrointestinal intolerance, based on the finding that coagulation occurs to a lesser degree than when a whey protein composition was used which has been subjected to extensive heat treatment. In case the protein composition further comprises casein, the positive effects of native whey proteins are preserved when the casein is beta-casein, i.e. when the composition is substantially devoid of alpha- and kappa-casein, while the presence of alpha-casein leads to increased protein coagulation.

The reduction in and/or prevention of the occurrence of gastrointestinal intolerance caused by the native whey protein composition according to the invention, occurs when compared to the whey protein that are typically present in nutritional compositions such as infant formula, which are not native or native to a much lesser extent. Thus, treatment and/or prevention of gastrointestinal intolerance and symptoms thereof is preferably understood to imply that the occurrence of gastrointestinal intolerance in a subject is reduced or prevented to a greater extent compared to the occurrence of gastrointestinal intolerance if the subject is fed the same whey protein composition wherein the whey protein is non-native whey protein, preferably by sufficient heating to reach a nativity of whey protein of less than 30%.

The application of the present invention is typically medical in nature. Hence, in a preferred embodiment, the subject is suffering from gastrointestinal intolerance or is at increased risk of developing gastrointestinal intolerance. In a preferred embodiment, the native whey protein according to the invention is for (a) treatment and/or prevention of delayed or slow gastric emptying; (b) reduction of the rate and/or extent of coagulation in the stomach; (c) increase of the rate of gastric emptying; (d) improvement of tolerance in the upper gastrointestinal tract; (e) improvement of intestinal transit. In a preferred embodiment, the native whey protein according to the invention is at least for one or more of (a) -(c).

Gastrointestinal intolerance is known to occur at a higher incidence in infants fed classical infant formula with non-native whey protein as compared to infants fed human breast milk. Gastrointestinal intolerance is characterized by the occurrence of increased gastric residuals, delayed gastric emptying, abdominal distension, regurgitation, colics, reflux, emesis and/or diarrhea. Gastrointestinal intolerance may result in malabsorption, impaired growth.

At particular risk for gastrointestinal intolerance are preterm infants. The invention is particularly directed to preterm infants. In a preferred embodiment, the invention is preferably directed to prevent or reduce regurgitation or reflux in infants, particularly preterm infants.

The native whey protein composition according to the invention improves gastric emptying and reduces the occurrence of gastric residuals and gastric residual volume. The native whey protein composition reduces the formation of coagulates in the stomach. Coagulation of proteins in the upper gastro-intestinal tract, in particular in the stomach, is hypothesised to delay gastric emptying. This can result in upper gastrointestinal complications like reflux, regurgitation, gastrointestinal discomfort and nausea, but also to confer satiety and the feeling of having a full stomach when this is not intended yet. The native whey protein composition is of particular use to prevent and/or treat reflux, regurgitation, gastrointestinal discomfort and nausea, most preferably to prevent and/or treat reflux and/or regurgitation. The effect on the prevention and/or treatment of reflux and/or regurgitation with the native whey protein composition is with respect to the occurrence thereof in a subject fed the same whey protein composition which is made non-native

The possibility to reduce digestive coagulation allows to prevent and treat upper gastrointestinal-related conditions such as intestinal discomfort, reflux, regurgitation, colics, high gastric residual volume (GRV), vomiting, nausea, bloating, and delayed gastric emptying. Further, facilitating gastric emptying is desired when aiming to promote digestive comfort. Thus, in one embodiment, the invention is directed at the treatment and/or prevention of one or more of these disorders.

Consumption of the native whey protein composition according to the invention serves to lower coagulate particle size and/or reduce the number of coagulate particles in the stomach. This is expected to improve peptic digestion and gastric emptying of the stomach content. Thus, the invention further relates to the use of native whey protein composition according to the present invention, such as obtainable by the process according to the present invention, in the reduction or preferably prevention of protein coagulation in the upper gastrointestinal tract, in particular the stomach, of the subject. The native whey protein composition is preferably for use in preventing and/or reducing the rate and extent of coagulation in the stomach. In an embodiment the native whey protein composition is for use in preventing and/or reducing the rate and extent of coagulation in the stomach of a preterm infant.

Coagulation and digestibility are different parameters. Gastrointestinal intolerance is also not a food allergy.

In an embodiment the native whey protein composition is for use in improving gastric emptying and/or for use in the treatment and/or prevention of delayed or slow gastric emptying. The native whey protein composition may further be for use in increasing the rate of gastric emptying. The effect on gastric emptying with the native whey protein composition according to the invention is with respect to the occurrence of gastric emptying in a subject fed the same whey protein composition which is made non-native.

In the context of the present invention, the native whey protein composition, typically as comprised in a nutritional composition as defined above, is administered to a subject at risk thereof and/or in need thereof. The subject is typically an infant, preferably a human infant. Preferably, the infant is selected from the group consisting of preterm infants, infants that are small for gestational age, infants with a low birth weight, very young infants and/or infants suffering from reflux and/or mild regurgitation. Preferably, the infant is 0-36 months of age, more preferably 0-12 months of age, even more preferably 0-6 months of age.

In a preferred embodiment, the subject is in need of reducing and/or preventing gastrointestinal intolerance. In one embodiment, the subject suffers from delayed or slow gastric emptying. In one embodiment, the subject is in need of a reduction of gastric coagulation. In one embodiment, the subject is at risk of developing gastrointestinal intolerance, more in particular a preterm subject suffering from or at risk of gastrointestinal intolerance.

In particular, the subject in need of reducing gastrointestinal intolerance or suffering from gastrointestinal intolerance is an infant selected from the group consisting of preterm infants, infants that are small for gestational age, infants with a low birth weight, very young infants and/or infants suffering from reflux and/or mild regurgitation. A preterm infant, or premature infant, herein refers to an infant born before the 37th week of gestation. An infant that is small for gestational age [SGA] is an infant whose birth weight lies below the 10th percentile for that gestational age. Reasons for SGA can be several; for example, term or preterm infants can be born SGA because they have been the subject of intrauterine growth restriction (IUGR). Many preterm infants are also small for gestational age. Premature and/or SGA infants include low birth weight infants (LBW infants), very low birth weight infants (VLBW infants), and extremely low birth weight infants (ELBW infants). LBW infants are infants with a birth weight below 2500 g; this group includes term infants born SGA. Very young infants herein refers to infants that are 0-6 months of age, preferably 0-3 months of age, more preferably 0-1 months of age.

The nutritional composition according to the invention is typically suitable as complete nutritional product for infants, like regular infant formula, regular follow-on formula or regular preterm formula. This means that the present nutrition composition is not or does not consist of human milk. Administration of the infant formula according to the invention this occurs as (part of) the regular feeding regime of the infant. In one embodiment, the use according to the invention is further for providing nutrition to the infant.

The nutritional composition comprises 3 to 7 g lipid/100 kcal, preferably 4 to 6 g lipid/100 kcal, more preferably 4.5 to 5.5 g lipid/100 kcal, 1.25 to 5 g protein/100 kcal, preferably 1.35 to 4 g protein/100 kcal, more preferably 1.5 to 3 g protein/100 kcal, more preferably 1.25 to 2.5 g protein/100 kcal, more preferably 1.25 to 2.25 g/100 kcal, even more preferably 1.25 to 2.1 g protein/100 kcal and 6 to 18 g digestible carbohydrate/100 kcal, preferably 8 to 16 g digestible carbohydrate/100 kcal, more preferably 10 to 15 g digestible carbohydrate/100 kcal.

In one aspect the nutritional composition selected from a preterm formula, an infant formula and a follow on formula comprises 3 to 7 g lipid/100 kcal, 1.25 to 5 g protein/100 kcal and 6 to 18 g digestible carbohydrate/100 kcal wherein the protein fraction consists of the native whey protein composition.

Associated with the method and use here above, the invention also pertains to a nutritional composition selected from a preterm formula, an infant formula and a follow-on formula comprising 3 to 7 g lipid/100 kcal, 1.25 to 5 g protein/100 kcal and 6 to 18 g digestible carbohydrate/100 kcal wherein the protein fraction consists of whey protein and beta-casein, and is substantially devoid of alpha-casein and kappa-casein, preferably comprising less than 6 wt %, preferably less than 4 wt %, more preferably less than 2 wt % of the sum of alpha-casein and kappa-casein, based on the total protein weight of the protein fraction, and wherein the formula optionally comprises added free amino acids, wherein the ratio of whey protein to beta-casein is in the range of between 85:15 and 55:45, preferably between 80:20 and 55:45, more preferably between 80:20 and 60:40, more preferably between 80:20 and 70:30, wherein the protein fraction has been pasteurized, and wherein the whey protein has a nativity of more than 80%, preferably more than 90%, more preferably more than 92%, preferably more than 94%, preferably more than 95%, even more than 98% of the whey protein. Preferably the nutritional composition has a beta-lactoglubulin: alpha-lactalbumin weight ratio not exceeding 70:30. The formula may optionally be supplemented with free amino acids, preferably less than 2 wt % of all proteinaceous matter. The preferred free amino acids are tyrosine and cysteine, preferably at least tyrosine, in an amount from 0.1 to 2 wt % based on the total weight of protein in the formula. Depending on the amount of protein in the formula, these amino acids may be supplemented to improve the amino acid profile in accordance with amino acid requirements for formulas.

The invention thus concerns a native whey protein composition for use in reduction in and/or prevention of the occurrence of gastrointestinal intolerance. The reduction in and/or prevention of the occurrence of gastrointestinal intolerance preferably involves
(a) treatment and/or prevention of delayed or slow gastric emptying; and/or
(b) reduction of the rate and/or extent of coagulation in the stomach; and/or
(c) increase the rate of gastric emptying; and/or
(d) improvement of tolerance in the upper gastrointestinal tract; and/or
(e) improvement of intestinal transit.

The invention particularly concerns a native whey protein composition for use in reduction in and/or prevention of the occurrence of gastrointestinal intolerance in a preterm infant. The reduction in and/or prevention of the occurrence of gastrointestinal intolerance preferably involves (a) treatment and/or prevention of delayed or slow gastric emptying; and/or
(b) reduction of the rate and/or extent of coagulation in the stomach; and/or
(c) increase the rate of gastric emptying; and/or
(d) improvement of tolerance in the upper gastrointestinal tract; and/or
(e) improvement of intestinal transit in said preterm infant.

The invention also concerns a native whey protein composition for use in the treatment and/or prevention of delayed and or slow gastric emptying.

The invention also concerns a native whey protein composition for use in reducing the rate and extent of coagulation in the stomach.

The invention also concerns a native whey protein composition for use in increasing the rate of gastric emptying.

The invention also concerns a native whey protein composition for use in improving intestinal transit. The invention also concerns a native whey protein composition for use in the prevention and/or treatment of regurgitation and/or reflux.

The invention also concerns a (non-therapeutic) method for administering a native whey protein composition for reducing the rate and/or extent of coagulation in the stomach; and/or increasing the rate of gastric emptying; and/or improving intestinal transit in a subject, preferably a subject that is not at imminent or at increased risk of gastrointestinal intolerance.

The invention as defined above is equally applicable to composition for use according to the invention, the method according to the invention and the use according to the invention.

### DESCRIPTION OF THE FIGURES

FIG. 1. Clinical observations piglet study. (a-c) Fecal scores of preterm and near-term piglets at (a) day 4 at 9 am, (b) day 4 at 6 pm and (c) day 5 at 9 am. (d-g) Gastric aspirates were taken 3 h after a bolus of EN and volume (ml) was measured at (d) day 1, (e) day 2, (f) day 3 and (g) day 4. (h-i) n=14 for preterm piglets, n=8-9 for near-term piglets. *p<0.05, **p<0.01, ns=non-significant based on t-test between EH-WPC and MP-WPC.

FIG. 2. GI transit. (a-d) Weight of small intestine and colon content was determined at day 5 (g/kg bodyweight), 1 h after the last bolus of EN, in separated regions (a) proximal small intestine, (b) mid small intestine, (c) distal small intestine and (d) colon. n=14 for preterm piglets, n=8-9 for near-term piglets. *p<0.05, **p<0.01, ns=non-significant based on Mann-Whitney test between EH-WPC and MP-WPC.

FIG. 3. Digestion & coagulation in vivo. (a) gastric content volume and (b) relative weight of the empty stomach (g/kg bodyweight) were measured at day 5 during necropsy, 1 h after the last bolus of EN, with (c) pH levels of the gastric content. (d-e) correlation between gastric content volume and pH levels for (d) preterm and (e) near-term piglets. n=14 for preterm piglets, n=8-9 for near-term piglets. *p<0.05, **p<0.01, ns=non-significant based on Mann-Whitney test between EH-WPC and MP-WPC. (f) Gastric content of 6 near-term piglets (n=3 per diet) was separated based on particle size and weight of separate fractions (g) was recorded. *p<0.05, **p<0.01, ns=non-significant based on t-test between EH-WPC and MP-WPC.

FIG. 4. Digestion & coagulation in vitro. (a) In vitro digested WPC was separated based on particle size and weight of the separate fractions (g) was recorded. (b) Protein concentration of the separated fractions determined by BCA. (c) Protein concentrations calculated based on SDS-PAGE separation for the coagulate with a particle size 1>D>0.25 mm. n=3, ND=not detectable, *p<0.05, **p<0.01, ns=non-significant based on t-test between EH-WPC and MP-WPC. BSA=bovine serum albumin, IgG (HC)=immunoglobulin G (heavy chain), βCAS=β-casein, βLG=β-lactoglobulin, αLA=α-lactalbumin.

FIG. 5 shows the results of Example 9 and demonstrates that a composition comprising native whey protein and beta-casein formed less coagulates than a composition comprising native whey protein and a mixture of alpha- and beta-casein under simulated gastric conditions.

### EXAMPLES

The following examples illustrate the invention.

### Example 1: WPC Preparation

Two whey protein compositions comprising whey protein and casein [WPC] products, (i) mildly-pasteurized WPC [MP-WPC], and (ii) extensively heat-treated WPC [EH-WPC], were prepared according to the following process. Milk and subsequent fractions were stored at 4° C. throughout production. Whole raw milk (purchased from Dairygold) was skimmed using typical GEA Westfalia Separator @ 55° C. and cooled to 4° C. Skim milk was subjected to cold [symbol] to separate casein from both whey and lactose. Microfiltration membrane used was a 0.08 M Synder membrane FR (PVDF 800 kDa) spiral wound membrane. The microfiltration retentate (MFR) was kept as the casein fraction and the microfiltration permeate (MFP) contained whey, β-casein, lactose and ash. The operating temperature was 10° C. and volume concentration factor (VCF) was 3. The MFP was then subjected to ultrafiltration to separate whey protein from lactose at operating temperature of 10° C. with VCF of 90. This VCF factor gave an optimal final concentration of whey protein in ultrafiltration retentate (UFR). A native WPC was produced. The ultrafiltration membrane used was a 10 kDa Synder membrane ST (PES 10 kDa) spiral wound membrane. Diafiltration medium was added to improve separation efficiency of membranes (200% of original starting skim milk volume). Concentrated liquid WPC (DM 11%) was stored at 4° C. until further handling. The WPC was heated to 30° C. and spray dried at 11% DM. The spray-dryer used was a single stage pilot scale dryer operated with an inlet temperature of 185° C. and outlet temperature of 90° C. This sample is referred to as native WPC.

The spray-dried WPC was then prepared to represent a mildly treated highly native, pasteurized protein sample which can be included in an infant formula. It was prepared by re-hydrating 100 g/L of native WPC in 40° C. RO water using a high speed mixer for 30 min, resulting in a total solids content of 10% and a protein content of about 7% (about 70 g protein per liter). This solution was heat-treated at 73° C./30 s using a MicroThermics tubular heat exchanger (MicroThermics, North Carolina, USA) and then freeze-dried resulting in MP-WPC powder with whey protein nativity of >95%, determined according to example 2.

MP-WPC powder was dissolved in demineralized water at 100 g/L, corresponding to 68.3 g protein per liter. pH was adjusted to 7.1 by addition of 1M NaOH, to prevent gelation during heating. Half of the MP-WPC solution was used without further processing (MP-WPC). The other part was thermally treated to denature whey protein using a shaking water bath at 80° C. for 20 min: extensively heated-WPC (EH-WPC). Temperature was monitored and reached 80° C. after 14 min, thus the effective thermal treatment at 80° C. was 6 min. The EH-WPC was then freeze-dried resulting in a WPC powder with whey protein nativity of <30%.

### Example 2: Soluble Protein Fraction Determination and Nativity Calculation

To determine the soluble protein fraction, WPC protein solution at pH 7.1 was centrifuged at 15000×g for 30 min and the supernatant was collected for protein quantification. The level of protein denaturation was determined by precipitation of the aggregated and unfolded proteins at pH 4.6 (adjusted by 0.1 M HCl) followed by centrifugation at 15000×g for 30 min to collect the supernatant as described by: Delahaije, R. J Journal of agricultural and food chemistry 2016, 64, 4362-4370. Crude protein (N×6.25) quantification in the total protein solution, the soluble fraction and the native fraction was performed by DUMAS as described in Chibnall A C, The Biochemical journal 1943, 37, 354-359, and soluble and native protein were expressed as % (w/w) of total protein.

Extensive heating only mildly affected protein solubility at pH 7.1, with 98% for MP-WPC compared with 87% for EH-WPC. In contrast, protein solubility at pH 4.6 was highly affected by the extensive heat treatment, with 72% for MP-WPC compared with 21% for the EH-WPC.

Since all milk proteins except native whey proteins precipitate at pH 4.6 and between 24-28% of the protein in the WPC fractions was β-casein (table 1), it follows that the whey protein fraction can be considered close to 100% native in the MP-WPC and only 30% native in EH-WPC.

The total nitrogen (TN), non-protein nitrogen (NPN) and non-casein nitrogen (NCN) were determined via Kjeldahl analysis, as per the ISO 8968-3/IDF 20-3:2004 standard (Milk—Determination of nitrogen content—Part 3: Block-digestion method (Semi-micro rapid routine method), 2004), using an automatic Kjeltec 8400 unit (FOSS, Warrington, U.K). The nativities of the whey proteins in Example 1 was calculated as follows:
(a) Casein fraction=(TP−NPN)−NCN
(b) Whey fraction=NCN−NPN
(c) Nativity=measured whey fraction (b)/theoretical whey faction*100%
   The theoretical whey fraction is based on the casein/whey protein ratio of the product, from the recipe of the product.

### Example 3: Protein Profile Analysis

The protein composition of both fresh and digested EH-WPC and MP-WPC was evaluated by SDS-polyacrylamide gel electrophoresis (SDS-PAGE) on NuPAGE™ 4-12% bis-tris midi protein gel (ThermoFisher Scientific, Amsterdam, the Netherlands). Equal levels of protein per sample were loaded, as determined by BCA reaction. Analysis was performed under reducing (with 2-mercaptoethanol (2ME)), and also under non-reducing conditions, to evaluate the protein composition of aggregates held together by disulfide bridges. As a result of heat treatment, disulfide bond formation can occur between two cysteine residues present in the proteins, creating aggregation of proteins. Proteins and peptides were identified using the PageRuler™ Plus protein ladder (10-250 kDa, #26619, ThermoFisher Scientific, Amsterdam, the Netherlands), and included bovine serum albumin (BSA) 69 kDa, immunoglobulin G heavy chain polypeptide (IgG HC) 53 kDa, β-casein ((3CAS) 24 kDa, (i-lactoglobulin ((3LG) 18 kDa and α-lactalbumin 14 kDa. Gels were scanned using Gel Doc Universal Hood II (Bio-rad, California, US) and densitometric analysis was performed in Quantity One version 4.6.9 (Bio-Rad, California, US). Each peak intensity displayed on the densitogram corresponds to the amount of soluble proteins that migrated through the gel to their specific protein size.

Both BSA and IgG (HC) were found to be aggregated, as these bands disappeared under non-reducing conditions for both EH-WPC and MP-WPC. BSA consists of mixed disulfides that can form polymers, which react very rapidly to give aggregates upon heat treatment, even after mild pasteurization only. IgG heavy chains are connected by disulfide bridges to form dimers in normal conformation. The effect of extensive heat treatment was most striking for α-lactalbumin and to a smaller extent also for β-lactoglobulin, with a decrease in band intensity under non-reducing conditions for EH-WPC only. This data implies that α-lactalbumin and β-lactoglobulin in the MP-WPC are present freely without a covalent link to other proteins, but upon extensive heating become a covalently linked part of protein aggregates/polymers. Beta-casein was found to be present at a weight percentage of about 24-28%, no other types of caseins were observed in the SDS-PAGE.

The protein composition in WPC was determined by SDS-PAGE analysis as percentage of the total protein.

TABLE 1
Protein composition whey protein compositions
comprising native whey protein and casein [WPC]

| % of total protein | Whey protein composition [WPC] |
|---|---|
| β-lactoglobulin | 37%-39% |
| α-lactalbumin | 20%-23% |
| β-casein | 24%-28% |
| Other whey proteins (a.o. BSA, IgG) | 13%-16% |

### Example 4: Protein Maillardation Determination

To gain insights in the degree of protein Maillardation, the level of carboxymethyllysine (CML), an advanced glycation end-product, was determined by UFLC/Flu method. Protein was hydrolysed in 6M hydrochloric acid, and CML in the hydrolysate was quantified in μg/ml by UFLC using a pre-column derivatization with o-phtaldialdehyde and fluorimetry as detection.

Compared with EH-WPC, there was a significantly lower level of CML detected in the MP-WPC; EH-WPC had a CML level of 18.8 μg/ml ±1.6 and the MP-WPC had a CML level of 16.1 μg/ml ±0.4 respectively.

### Example 5: In Vivo Effect on Gastrointestinal Tolerance

A MP-WPC whey protein product with high nativity of over 90%, obtained according to the process of Example 1 was analysed with respect to its properties to gastrointestinal intolerance prevention, gastric emptying and upper gastrointestinal coagulation behaviour. Gastric parameters in near-term and preterm piglets were compared to an identical whey protein product which was denatured to a nativity level of below 40% by extensive heating obtained according to the process of Example 1.

Piglet study: Preterm and near-term pigs (Danish Landrace×Large White×Duroc) were delivered from sows by caesarean section at 90% gestation (106 days of gestation, n=34, 2 litters) and 96% gestation (113 days of gestation, n=18, 1 litter) respectively. Piglets were transferred to the intensive care unit and housed individually in heated incubators with air and oxygen supply. Piglets were passively immunized and surgically prepared with an orogastric feeding tube and an arterial catheter for parenteral nutrition (PN). Piglets from each litter were block randomized according to birthweight into two groups of enteral diets: 1) EH-WPC group and 2) MP-WPC group. During the study, investigators were all blinded for type of diet. The Danish National Committee on Animal Experimentation approved all procedures, which is in accordance with the EU Directive 2010/63/EU Article 23.2 and the Danish executive order no 2014-15-0201-00418. Piglets received minimal enteral nutrition for 5 days based on EH-WPC or MP-WPC, via an orogastric tube (6F, Portex, UK). Each formula consisted of 80 g/L WPC, 50 g/L Pepdite (infant milk formula containing non-milk derived low molecular weight peptides, essential amino acids, carbohydrates, fats, vitamins, minerals and trace elements), 50 g/L Liquigen (medium-chain fatty acids) and 30 g/L Calogen (long-chain fatty acids) (all obtained from Nutricia advanced medical nutrition). Macronutrient composition of the formulas was as follows: 3629 kJ/L energy, 59 g/L protein, 52 g/L fat, 39 g/L carbohydrate (of which 21 g/L maltodextrin, 2.7 g/L maltotriose and 1.8 g/L maltose and 16 g/L lactose). During the study period, piglets received the enteral nutrition via the orogastric tube in increasing dose as indicated in Table 2 below, with additional continuous parenteral nutrition support (Kabiven, Fresenius Kabi) through an umbilical catheter. 1 h prior to euthanasia on day 5, the pigs received a last bolus EN of 15 ml/kg bodyweight. Additional enteral boluses via the orogastric tube included galactose on day 3, lactose and x-ray contrast fluid (Iodixnol, Visipaque) on day 4 and lactulose/mannitol at day 5, as indicated in Table 2.

TABLE 2
Feeding regimen

| | Enteral | Parenteral | |
|---|---|---|---|
| Time | Nutrition | Nutrition | Bolus |
| Day 1 | 6 ml/kg/3 h | 4 ml/kg/h | — |
| Day 2 | 8 ml/kg/3 h | 4 ml/kg/h | — |
| Day 3 | 8 ml/kg/3 h | 4 ml/kg/h | 5% galactose: 15 ml/kg |
| Day 4 | 10 ml/kg/3 h | 4 ml/kg/h | 10% lactose: 15 ml/kg |
| Day 5 | 10 ml/kg/3 h | 4 ml/kg/h | 5% lactulose/mannitol: 15 ml/kg |
| | | | 3 hrs. before euthanasia |
| | | | last bolus of EN: 15 ml/kg |
| | | | 1 hr before euthanasia |

Clinical evaluation and sample collection: Pigs were continuously monitored for clinical symptoms of feeding intolerance, vomiting, abdominal distention, haemorrhagic diarrhoea and/or respiratory distress during the entire study. Gastric residuals were measured and sampled to investigate the value of gastric residual as a marker of feeding tolerance. Aspirates were taken three times a day, prior to EN feeding (i.e., 3 h after the previous bolus). One ml of air was put into the feeding tube, after which gastric content was pulled back up until vacuum was reached. The total volume of gastric aspirate was measured and, when possible, 1 ml of aspirate was collected and stored at −80° C. Remaining gastric aspirate was returned into the stomach gently via the tube. Fecal assessment was performed twice a day based on stool frequency and stool consistency, according to Table 3 below. Both clinical and fecal assessment were performed by personnel blinded for the diet.

TABLE 3
Fecal assessment. Fecal assessment was performed twice a
day blinded for diet. Date and time for first meconium
after birth were also registered. For analysis, score ≥3
was classified as a piglet with diarrhoea.

| Score | Feces |
|---|---|
| 0 | no stool |
| 1 | firm feces |
| 2 | pasty feces |
| 3 | Droplets of watery feces/diarrhoea |
| 4 | Moderate amounts of diarrhoea |
| 5 | Large amounts of diarrhoea |

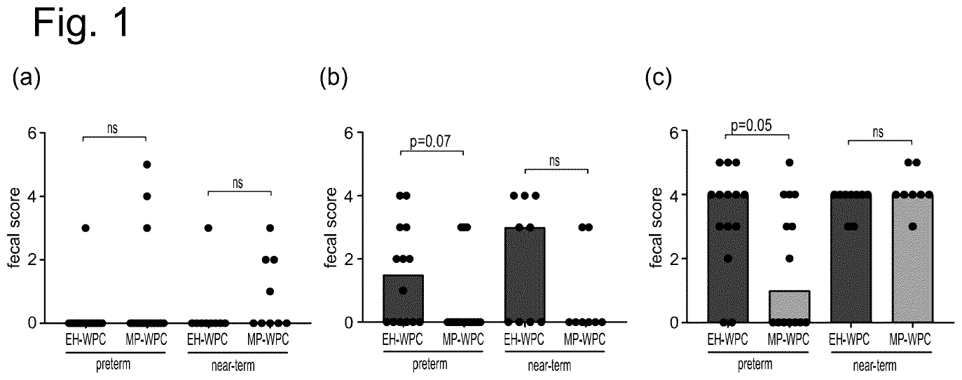

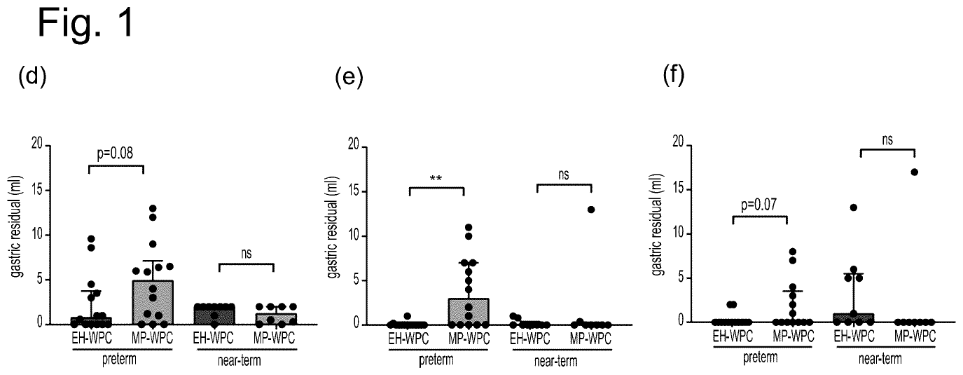

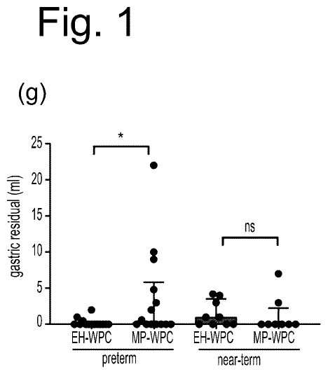

Fecal scores, based on stool frequency and consistency (Table 3), showed minimal to no feces on day 1, 2 and 3 for any of the preterm or near-term piglets, but an extensive increase on days 4 and 5 (FIG. 1a-c). The overall increase from 9 am to 6 pm at day 4 was potentially induced by an oral lactose challenge (data not shown), while the increase from day 4 to 5 might be the result of the x-ray contrast fluid. With the increasing fecal scores, there was a strong trend towards lower fecal scores for preterm piglets receiving MP-WPC compared with EH-WPC on day 4 at 6 pm (FIG. 1b) and day 5 at 9 am (FIG. 1c), suggesting improved GI tolerance in preterm piglets fed MP-WPC. In near-term piglets, aspirate volumes were low and not significantly different between MP-WPC and EH-WPC on all days (FIG. 1d-g).

An important indicator of feeding intolerance in preterm infants used in the clinic is the presence of gastric residuals, which is assessed by taking gastric aspirates. During this study, remarkable differences in gastric residual volumes 3 h after an enteral bolus were observed. In general, the volume of gastric residuals decreased over the days, with detectable gastric residuals in most piglets at day 1 (FIG. 1d) but undetectable gastric residuals in most piglets at day 4 (FIG. 1g).

In preterm piglets, the volume of gastric residuals was higher in the piglets receiving MP-WPC compared with EH-WPC on all days (FIG. 1d-g), suggesting that either the MP-WPC receiving piglets had more diet left in the stomach, or that the EH-WPC residuals could not be aspirated via the small diameter of the tube. Since opposite results were found for the gastric content/residual volume present at necropsy (discussed below), likely, the coagulated gastric content could not be aspirated via the feeding tube and, thereby, impaired accurate evaluation of the gastric residuals by aspiration.

### GI Transit by X-Ray Analysis

On day 4 in the evening, gut transit time was assessed by x-ray photography after oral intake of a contrast solution, as described previously by Chen, Pediatric Research, 2020. Each piglet received an enteral bolus of 4 ml/kg contrast fluid (4 ml/kg, Idoixnol, Visipaque®, GE Healthcare, Brondby, Denmark) after 2 h of fasting, to mimic clinical practices. X-ray images of the GI tract were taken using Mobilett XP Hybrid (Siemens, Germany) at 20 min, 1, 2 and 4 h and subsequent every second hour till the contrast fluid was cleared from the GI tract or maximally 14 hours, which ever came first. The piglets were placed on their back while the x-ray was taken and returned to their home cage between screenings. During the x-ray examination, piglets received enteral nutrition according to the regular feeding schedule (i.e., every 3 h). X-ray images were interpreted by both a neonatologist and a radiologist, blinded to the types of diet. For analysis, time of contrast to be cleared from the stomach (StEmpty), to be cleared from the small intestine (SlEmpty), to first appear at caecum (ToCaecum) and to first appear in the rectum (ToRectum) were recorded.

When assessing stomach emptying (StEmpty) of contrast fluid by x-ray on day 4, there were no differences between MP-WPC and EH-WPC in either preterm or near-term piglets (data not shown). The contrast fluid was emptied from the stomach in similar rate for both diets, suggesting that the differences in gastric emptying observed were mainly caused by the coagulation and not due to differences in motility.

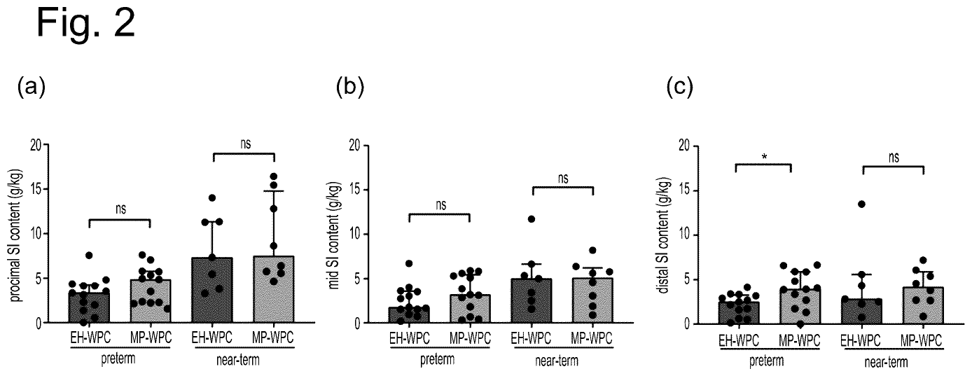

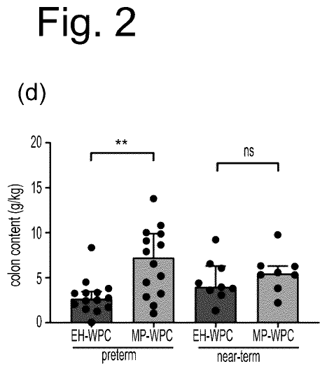

Coagulation of proteins can delay gastric emptying and impact GI transit, therefore the small intestine (SI) and colon content was determined (FIG. 2a-d). The volume of the distal SI content (FIG. 2c) and colon content (FIG. 2d) were significantly higher in the preterm piglets fed MP-WPC compared with those fed EH-WPC based formula, suggesting a significant effect on intestinal transit in preterm.

### Sample Collection

Piglets were euthanized on day 5 for sample collection, or when clinical symptoms of feeding intolerance, vomiting, abdominal distention, haemorrhagic diarrhoea and/or respiratory distress appeared during the study and the humane endpoint was reached. The piglets were first anaesthetized with an intramuscular injection of Zoletil mix (0.1 mL/kg, Virbac, Kolding, Denmark) and subsequently mL of 20% pentobarbital (Euthanimal, Scanvet, Denmark) was injected intracardiac to euthanize the piglet. Weight of full and empty stomach was recorded to determine the volume of the gastric content, and samples of gastric content were collected and stored at −80° C. until further analysis.

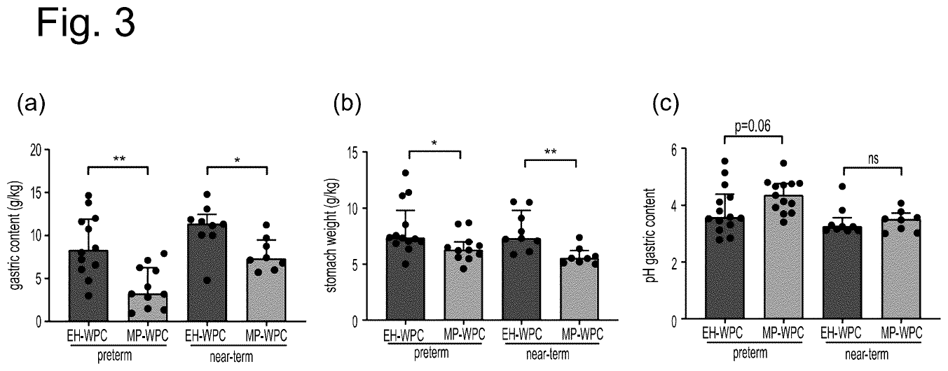

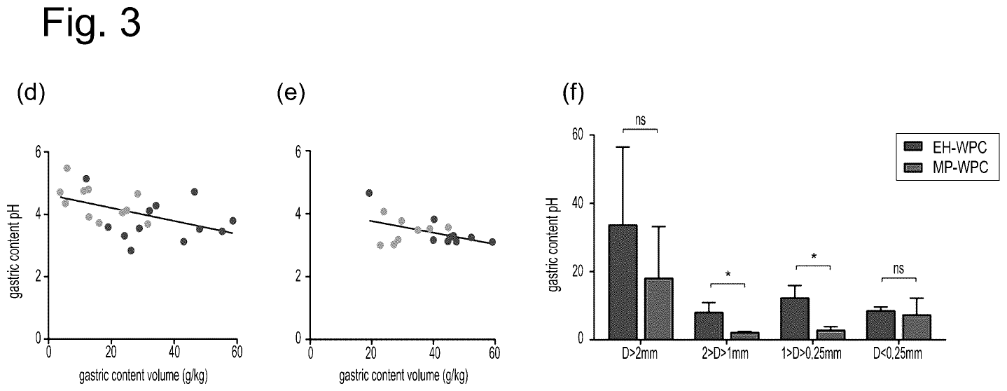

To decipher whether the MP-WPC was emptied slower from the stomach or whether the EH-WPC residual was too thick for aspiration via the gastric tube, the volumes of the gastric contents were measured during necropsy at day 5, 1 h after a 15 mL/kg bolus of EN. In contrast to the gastric aspirates, the volume of gastric contents was significantly lower in the piglets receiving MP-WPC compared with EH-WPC for both preterm and near-term piglets (FIG. 3a). Moreover, the volume of the gastric content was for most piglets higher than the volume of the last bolus administered, suggesting an additional effect of previous boluses. After collection of the content, the relative stomach weight of piglets receiving MP-WPC was lower compared with piglets receiving EH-WPC (FIG. 3b).

### Analysis of Digesta

To measure pH levels in the gastric content, 0.5 gram of gastric content was diluted in 1 ml of dH2O, vortexed and subsequently pH was recorded with the SensION+PH3 meter (HACH).

The gastric contents were poured over sequentially placed analytical sieves, with a mesh width of 2 mm, 1 mm and 0.25 mm (Retsch, VWR, Amsterdam, Netherlands). Coagulates were separated according to their particle diameter in four fractions: larger than 2 mm (D>2 mm), between 1 and 2 mm (2>D>1 mm), between 0.25 and 1 mm (1>D>0.25 mm) and the permeate which is smaller than 0.25 mm (D<0.25 mm). After 30 min, wet weight of the sieves and permeate was recorded to determine the wet weight of the fractions.

The pH levels of the gastric content tended to be higher in the MP-WPC compared with EH-WPC for preterm piglets (FIG. 3c), while this difference was less pronounced for the near-term piglets. There was a significant strong inverse correlation between gastric content volume and pH level for the preterm piglets (FIG. 3d, Pearson's r−0.49 p=0.02) and a similar trend in the near term piglets (FIG. 3e, Pearson's r−0.47 p=0.06). The increased gastric residual volume might have resulted in mechanical stimulation (distention) of receptors, which subsequently increase HCl secretion in the stomach. The lower pH subsequently may result in more coagulation of the proteins.

The gastric content of 6 near-term piglets (n=3 for each diet) were further analyzed to determine the formation and size of coagulates. The coagulates in the gastric content of piglets fed MP-WPC were smaller in weight and size compared with the coagulates in the gastric content of piglets fed EH-WPC (FIG. 31). The volume of the permeate (D<0.25 mm) was equal for both diets (FIG. 31).

Taken together, although the volumes of gastric aspirates were significantly higher, the gastric content were significantly lower in preterm piglets fed MP-WPC compared with EH-WPC, indicating that the gastric EH-WPC residuals were too thick for complete aspiration and thereby lead to an underestimation of the real gastric residual volume. Near-term piglets showed similar lower gastric content for MP-WPC, with a lower volume of coagulates in the gastric content present compared with EH-WPC.

### Example 6. In Vitro Gastric Digestion Assay

Digestion of the EH-WPC and MP-WPC was simulated in vitro in the semi-dynamic digestion model (SIM) described previously by van den Braak, Clinical Nutrition, 2013, 32, 765-771, with minor adaptations to mimic preterm infant digestion. In detail, gastric digestion was simulated over a period of 120 min at 37° C. in multi fermenter fed-batch bioreactors (Dasgip A G, Julich, Germany). Bioreactors were filled with a bolus of 150 ml WPC (100 g/L) and were mixed by short and gentle orbital shakings of the bioreactor every 10 min. At the start of the digestion, 25 ml of simulated saliva fluid (SSF) was added to each bioreactor. During the digestion, simulated gastric fluid (SGF) was added in a dynamic manner, with 9 ml in the first 3 min followed by a continuous addition of 22.5 ml/h. To decrease pH in time hydrochloric acid (1M, Sigma-Aldrich, Zwijndrecht, the Netherlands) was added in a pre-determined dynamic flow to result in a final pH of 4.3 after 120 min. SSF (pH 6.3) consisted of 0.1 M NaCl, 30 mM KCl (Merck, VWR International), 2 mM CaCl₂·2H₂O, 14 mM NaHCO₃, 0.06% (w/v) α-amylase (from Aspergillus oryzae, A9857) (all Sigma-Aldrich, Zwijndrecht, the Netherlands). SGF (pH 4.0) contained 50 mM NaCl, 15 mM KCl (Merck, VWR, Amsterdam, the Netherlands), 1 mM CaCl₂·2H₂O, 0.005% (w/v) pepsin (from porcine gastric mucosa, P7125), 0.013% (w/v) lipase (from Rhizopus oryzae, 80612) (all Sigma-Aldrich, Zwijndrecht, the Netherlands).

In vitro obtained digesta were poured over sequentially placed analytical sieves, with a mesh width of 2 mm, 1 mm and 0.25 mm (Retsch, VWR, Amsterdam, Netherlands). Coagulates were separated according to their particle diameter in four fractions: larger than 2 mm (D>2 mm), between 1 and 2 mm (2>D>1 mm), between 0.25 and 1 mm (1>D>0.25 mm) and the permeate which is smaller than 0.25 mm (D<0.25 mm). After 30 min, wet weight of the sieves and permeate was recorded to determine the wet weight of the fractions. Protein concentration in the separated fractions was measured by BCA

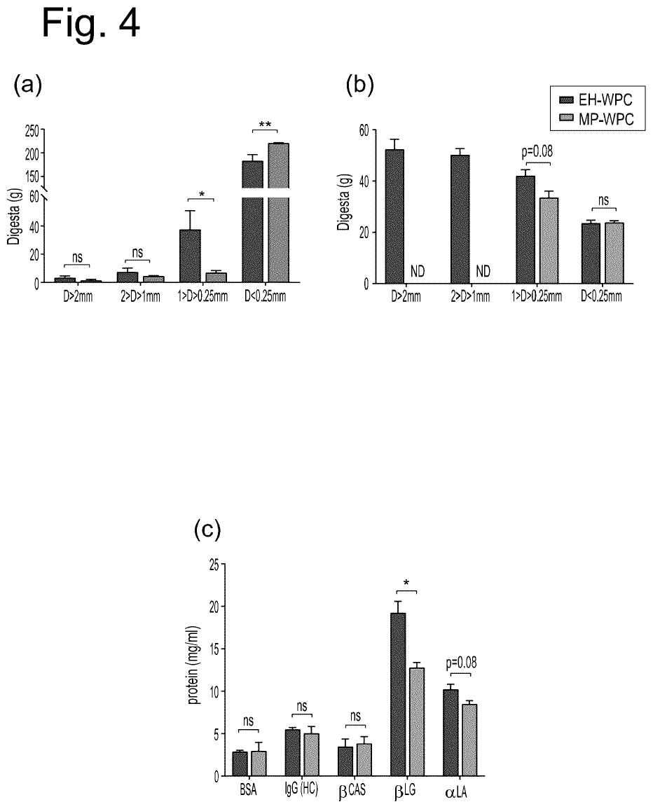

After 2 h of in vitro digestion, the digesta were poured over sequentially placed sieves separating the digesta based on particle size. Coagulates >1 mm were nearly undetectable in the gastric digesta of MP-WPC nor EH-WPC (FIG. 4a). There were significantly fewer particles with a size between 0.25 and 1 mm for the MP-WPC compared with EH-WPC, while the weight of the permeate (i.e., D<0.25 mm) was significantly larger in the MP-WPC compared with EH-WPC (FIG. 4a).

It was impossible to sample coagulates with size >2 mm and 2-1 mm for MP-WPC due to minimal digesta retained on the sieves. However, protein concentrations of the coagulated fraction 1>D>0.25 mm tended to be lower for MP-WPC compared than EH-WPC (FIG. 4b). Analysis of the protein composition in the different fractions analyzed by SDS-PAGE identified similar patterns between MP-WPC and EH-WPC. Based on protein concentrations measured in the separated fractions and the band intensity detected by SDS-PAGE, the concentration of specific proteins was calculated. Coagulates that were formed had comparable protein composition for both EH-WPC and MP-WPC. As expected, the relative abundance of β-casein was higher in the coagulate compared with the permeate, since this protein precipitates at low pH. As a consequence, the relative abundance of β-lactoglobulin, the most abundant whey protein, was lower in the coagulate compared with the permeate.

Comparing the coagulates from MP-WPC with the coagulates from EH-WPC revealed significant differences in protein composition. The concentration of β-lactoglobulin in the coagulates with particle size 1>D>0.25 mm was significant lower in digested MP-WPC compared with digested EH-WPC (FIG. 4c), with the same tendency for α-lactalbumin. This is most likely a result of the lower protein aggregation observed for MP-WPC and further underlines the relation between WPC heating and formation of gastric coagulates. Bovine serum albumin, immunoglobulin G (HC) and β-casein were present in these coagulates in similar concentrations for both MP-WPC and EH-WPC. In the permeate (D<0.25 mm), there were no differences in protein concentration detected between MP-WPC and EH-WPC for any of the specific proteins.

In summary, MP-WPC shows lower level of protein coagulation after in vitro simulated digestion compared with EH-WPC, with lower levels of β-lactoglobulin and α-lactalbumin in the coagulates.

### Example 7. Infant Formula Containing Native Whey Protein Concentrate (WPC)

An infant formula containing the native whey protein composition according to the invention is exemplified as follows. The main nutrients of this infant formula are as follows:

| | Units | Per 100 ml RTF | Per 100 kcal |
|---|---|---|---|
| Energy value | kcal | 66 | 100 |
| Protein | g | 1.3 | 2 |
| Whey | g | 0.94 | 1.44 |
| beta-casein | g | 0.36 | 0.56 |
| free tyrosine | mg | 7.92 | 12 |
| Weight ratio | 64:36 | | |
| β-lactoglobulin:α-lactalbumin | | | |
| Carbohydrate | g | 7.3 | 11.1 |
| of which sugars | g | 7.2 | 10.9 |
| Glucose | g | 0.2 | 0.3 |
| Lactose | g | 7.0 | 10.6 |
| Galactose | g | 0.01 | 0.02 |
| Polysaccharides | g | 0.01 | 0.02 |
| Fat | g | 3.4 | 5.1 |
| Vegetable | g | 3.3 | 5 |
| Animal | g | 0.1 | 0.1 |
| Saturated | g | 1.5 | 2.2 |
| Monounsaturated | g | 1.4 | 2.1 |
| Polyunsaturated | g | 0.6 | 0.8 |

The infant formula is intended for feeding of term infants aged 0 to 6 months. In terms of energy value, the infant formula contains 8 En % protein, 44 En % carbohydrate, 46 En % fat. Minerals and vitamins and other micronutrients are included according to prevailing nutritional guidelines to produce a complete enteral infant feed. The indicated totals may not be reached due to rounding off of values. RTF=Ready-To-Feed. Whey protein is present in the infant formula with a nativity of more than 90%.

### Example 8. Preterm Formula Containing Native Whey Protein Concentrate (WPC)

A preterm formula containing the native whey protein composition according to the invention is exemplified as follows. The main nutrients of this preterm formula are as follows.

| | Units | Per 100 ml RTF | Per 100 kcal |
|---|---|---|---|
| Energy value | kcal | 78 | 100 |
| Protein | g | 2.6 | 3.3 |
| Whey | g | 1.87 | 2.37 |
| beta-casein | g | 0.56 | 0.92 |
| Carbohydrate | g | 8.2 | 10.4 |
| of which sugars | g | 6.1 | 7.7 |
| Glucose | g | 0.3 | 0.4 |
| Lactose | g | 5.5 | 6.9 |
| Maltose | g | 0.2 | 0.3 |
| Polysaccharides | g | 2.1 | 2.6 |
| Fat | g | 3.8 | 4.8 |
| Vegetable | g | 3.4 | 4.2 |
| Animal | g | 0.3 | 0.5 |
| Saturated | g | 1.6 | 2.0 |
| Monounsaturated | g | 1.4 | 1.8 |
| Polyunsaturated | g | 0.8 | 1.0 |
| Minerals | g | 0.2 | 0.2 |

The preterm formula is intended for feeding of preterm infants, meaning infants born before the 37^{th} week of gestation. The protein amount is increased compared to infant formula intended for feeding of term infants for reasons related to catch-up growth which is intended to occur in preterm-born infants. In terms of energy value, the preterm formula contains 13 En % protein, 42 En % carbohydrate and 44 En % fat. RTF=Ready-To-Feed. Minerals and vitamins and other micronutrients are included according to nutritional guidelines to produce a complete enteral preterm feed. The indicated totals may not be reached due to rounding off of values. Whey protein is present in the preterm formula with a nativity of more than 90%.

### Example 9. In Vitro Gastric Digestion Assay

Gastric physicochemical behaviour of a native whey protein concentrate, wherein 30% of the protein was beta-casein (WPC-B) was compared to a native whey protein concentrate wherein 30% of the protein was a mixture of alpha- and beta-caseins (WPC-AB) and a whey protein isolate containing only whey proteins (WPI). Gastric simulation tests were performed in duplicate. Pre-term infant gastric conditions were simulated by the method of Example 6. Multi-fermenter fed-batch bioreactors (Dasgip A G, Jülich, Germany) were placed at 37° C., filled with a bolus of 150 ml WPC/WPI (62.5 g/L) and were mixed by short and gentle orbital shakings of the bioreactor. Simulated gastric electrolyte (SGE) was added in one shot of 100 ml, afterwards the pH was decreased to pH 4.3 in twenty minutes using hydrochloric acid (1M) under magnetic stirring (150 rpm). SGE (pH 4.0) contained 50 mM NaCl, 15 mM KCl and 1 mM CaCl2·2H2O.

In vitro obtained digesta were poured over sequentially placed analytical sieves, with a mesh width of 2 mm, 1 mm and 0.25 mm (Retsch, VWR, Amsterdam, Netherlands). Coagulates were separated according to their particle diameter in three fractions: larger than 2 mm (D>2 mm), between 1 and 2 mm (2>D>1 mm) and between 0.25 and 1 mm (1>D>0.25 mm). After 30 min, wet weight of the sieves and permeate was recorded to determine the wet weight of the fractions.

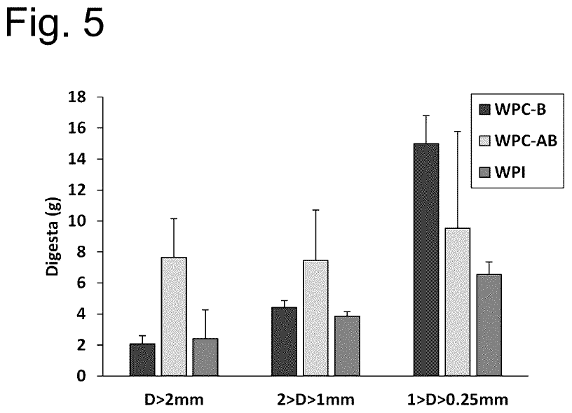

The results are shown in FIG. 5, and show that WPC-B formed less coagulates >2 mm and between 2 and 1 mm compared to WPC-AB and similar to WPI. In addition, the weight of the smallest coagulates, between 1 and 0.25 mm, was similar between WPC-B and WPC-AB, but higher than WPI. In conclusion, presence of beta-casein only in WPC inhibited formation of large particles compared to the presence of beta- and alpha-casein in WPC under in vitro gastric infant conditions.

22. A nutritional composition comprising 3 to 7 g lipid/100 kcal, 1.25 to 5 g protein/100 kcal and 6 to 18 g digestible carbohydrate/100 kcal,
wherein the protein fraction consists of whey protein and beta-casein, and is substantially devoid of alpha-casein and kappa-casein, and wherein the formula optionally comprises added free amino acids,
wherein the ratio of whey protein to beta-casein is in the range between 85:15 and 55:45, wherein the protein fraction has been pasteurized, and
wherein the whey protein has a nativity of more than 80%.

23. The nutritional composition according to claim 22, comprising less than 6 wt % of the sum of alpha-casein and kappacasein, based on the total protein weight of the protein fraction.

24. The nutritional composition according to claim 22, wherein the ratio beta-lactoglobulin to alphalactalbumin is below 7:3.

25. The nutritional composition according to claim 22, wherein the ratio of whey protein to beta-casein is in the range between 80:20 and 60:40.

26. The nutritional composition according to claim 22, wherein the whey protein has a nativity of more than 90%.

27. The nutritional composition according to claim 22, wherein the nutritional composition is selected from a preterm formula, an infant formula and a follow-on formula.

28. The nutritional composition according to claim 22, wherein the native whey protein composition has a protein solubility of more than 55% based on the total amount of protein in the native whey protein composition at acidic pH conditions.

29. The nutritional composition according to claim 22, wherein the native whey does not originate from acid whey or from sweet whey.

30. The nutritional composition according to claim 22, wherein the native whey protein composition is obtainable by cold membrane-filtration based technology.

31. The nutritional composition according to claim 22, wherein the native whey protein composition has been pasteurized at 72-74° C. for 15 to 30 seconds.

32. The nutritional composition according to claim 22, wherein the nutritional composition exhibits an alkaline phosphatase activity of at most 350 mU/L.

33. The nutritional composition according to claim 22, wherein the native whey protein composition and optionally at most 2 wt % of added free amino acids based on the total weight of protein in the nutritional composition are the sole protein sources for the nutritional composition.

34. The nutritional composition according to claim 22, wherein the native whey protein is obtainable by a process comprising:
(a) processing defatted milk into a casein stream, a whey protein stream and a lactose stream, by:
subjecting the defatted milk to a debacterialization treatment, to provide a debacterialized milk;
(ii) subjecting the debacterialized milk originating from step (i) to cold microfiltration over a membrane capable of retaining casein and permeating whey proteins, to provide a casein stream as retentate and a permeate comprising whey protein and β-casein;
(iii) fractionating the permeate originating from step (ii) into a whey protein stream comprising 1) whey protein and β-casein and 2) a lactose stream;
(b″) optionally spray-drying the whey protein stream originating from (a)-(iii) followed by dissolving;
wherein at least one of the debacterialization treatment of step (a)-(i) or the stream originating from step (a)-(iii) after optional step (b″) is subjected to pasteurization; and;
(c″) optionally freeze-drying the stream,
and wherein the debacterialization treatment (i) if not pasteurization, involves subjecting the defatted milk to microfiltration over a membrane capable of retaining bacteria, wherein the debacterialized milk is in the permeate.

35. The nutritional composition according to claim 34, wherein step (iii) is performed by ultrafiltration over a membrane capable of retaining whey proteins and permeating lactose, to provide a whey protein stream as retentate and a permeate comprising lactose.

36. The nutritional composition according to claim 35, wherein ultrafiltration step (iii) operates with a volume concentration factor in the range of 20-200.

37. A method for administering a nutritional composition comprising beta-casein and whey protein for reducing the rate and/or extent of coagulation in the stomach; and/or increasing the rate of gastric emptying; and/or improving intestinal transit in a subject that is not at imminent or at increased risk of gastrointestinal intolerance, wherein the nutritional composition is a composition according to claim 1, and wherein administration of the composition to the subject reduces the rate and/or extent of coagulation in the stomach; and/or increases the rate of gastric emptying; and/or improves intestinal transit in the subject.

38. The method according to claim 37, wherein administration of the composition to the subject prevents, reduces or treats regurgitation and/or reflux.

39. The method according to claim 34, wherein the subject is an infant selected from preterm infants, infant that is small for gestational age, infant with a low birth weight, very young infant and/or infant suffering from reflux and/or mild regurgitation.
